(19) United States
(12) Patent Application Publication
(10) Pub. No.: US 2003/0077273 A1
(43) Pub. Date: Apr. 24, 2003

(54) Methods of treating antibody-mediated pathologies using agents which inhibit CD21

(76) Inventors: Matthew D. Linnik, Solana Beach, CA (US); Mary-Ann Campbell, San Diego, CA (US)

Correspondence Address: Catherine M. Polizzi, Morrison & Foerster LLP, 755 Page Mill Road, Palo Alto, CA, 94304-1018, US

(21) Appl. No.: 10/150,469

(22) Filed: May 17, 2002

Related U.S. Application Data
(60) Provisional application No. 60/292,132, filed on May 17, 2001.

(51) Int. Cl. A61K 39/395

(52) U.S. Cl. 424/131.1; 424/144.1

(57) ABSTRACT

Provided herein are methods of treating antibody-mediated pathologies such as autoimmune diseases (e.g., SLE, ITP, and thyroiditis) using agents which inhibit CD21/C3d interaction to ameliorate one or more symptoms of an antibody-mediated pathology. Also provided herein are methods for delaying the development of antibody-mediated pathologies using agents that inhibit CD21/C3d interaction to effect such delay.

### CROSS REFERENCE TO RELATED APPLICATION

This application claims the benefit of U.S. provisional application serial No. 60/292,132, filed May 17, 2001, which is incorporated in its entirety by reference.

### TECHNICAL FIELD

This invention relates to the field of antibody-mediated pathologies such as autoimmune disease. More specifically, the invention relates to methods of treating individuals with antibody-mediated pathologies such as autoimmune disease and methods of delaying development of antibody-mediated pathologies using an agent which interferes with CD21/C3d interaction.

### BACKGROUND OF THE INVENTION

Antibody-mediated pathologies encompass a number of disorders which include autoimmune disease, xenograft rejection, allograft rejection, graft-versus-host disease, and immune response to therapeutic proteins that are administered continuously. Antibody-mediated autoimmune pathologies are characterized by excessive production of autoantibodies that can result in immune complexes. Deposits of immune complexes in specific organs or tissue sites can cause pathologies such as tissue damage, renal failure, glomerulonephritis, vasculitis, and severe organ involvement with pericarditis. Examples of antibody-mediated autoimmune pathologies include primary anti-phospholipid syndrome (APS), thyroiditis, myasthenia gravis, Graves' disease, systemic lupus erythematosus (SLE), systemic scleroderma, idiopathic thrombocytopenic purpura (ITP) and polymyositis.

Antiphospholipid (aPL) antibodies is the term generally given to describe autoantibodies that are associated with thrombosis, recurrent fetal loss and thrombocytopenia as the primary anti-phospholipid syndrome (APS) as well as autoimmune diseases such as systemic lupus erythematosus (SLE). Harris et al. (1983) Lancet 2:1211-1214; and Lockshin et al. (1985) N. Engl. J Med. 313:152-156. APS may be primary, or secondary, meaning that it is associated with other conditions, primarily SLE. “Phospholipid-Binding Antibodies” (Harris et al., eds., CRC Press, Boca Raton, Fla., 1991; McNeil et al. “Advances in Immunology”, Vol. 49, pp. 193-281 (Austen et al., eds., Academic Press, San Diego, Calif., 1991)). aPL antibodies include so-called anti-cardiolipin (aCL) autoantibodies, which are discussed below. aPL antibodies (including aCL antibodies) are detected in many conditions but only the β₂-glycoprotein I (β₂GPI-dependent antiphospholipid antibodies found in association with autoimmune disease require the presence of the phospholipid binding serum protein, β₂GPI. Vaarala et al. (1986) Clin. Immunol. Immunopathol. 41:8-15. The clinical manifestation of APS include arterial occlusion, extremity gangrene, stroke, myocardial infarct, other visceral infarct, venous occlusion, peripheral venous occlusion, visceral venous occlusion (e.g., Budd-Chiari syndrome, portal vein occlusion), recurrent fetal loss, thrombocytopenia, Coombs'-positive hemolytic anemia, livedo reticularis, neurological abnormalities (e.g., chorea, transient ischemic attacks), valvular heart disease, and sudden multisystem arterial occlusion. Scientific American Medicine Chapter 15 Section IV p. 5 (2001).

Thryroiditis (or Hashimoto's thyroiditis) and Graves' disease are other autoimmune diseases which involve the thyroid and are thought to be caused by autoantibodies to thyroid-stimulating hormone (TSH) receptor. Pathological findings in the thyroid include excessive infiltration with chronic inflammatory cells, follicular rupture, eosinophilia, varying degrees of hyperplasia, and fibrosis. The clinical manifestations of chronic thyroiditis are variable but major syndromes are painless goiter, hypothyroidism, and a combination of both. Scientific American Medicine Chapter 6 Section VI pp. 6-8 and Chapter 3 Section I p.16 (2001).

Myasthenia gravis is an autoimmune disease thought to be caused by autoantibodies to acetylcholine receptors (AchR). Autoantibodies to AchR can be detected and measured in serum of patients with myasthenia gravis. Clinical manifestations of myasthenia gravis include skeletal muscle weakness and fatigability. Muscle weakness can present as asymmetric ptosis and diplopia caused by the impaired ability to elevate the eyelids and movement of the extraocular muscles. Other physical symptoms include weak neck extensors, drooping of the head, facial snarl when patient attempts to smile due to weakness of facial and bulbar muscles, nasal or dysarthric and low-volume dysphonic speech, dysphagia which can result in choking or regurgitation, and skeletal muscle weakness which can cause difficulties in walking, climbing stairs, or carrying objects. The disease can be transmitted to experimental animals with patient's pathogenic IgG. Scientific American Medicine Chapter 6 Section VI pp. 6-8 and Chapter 11 Section III pp. 12-13(2001).

Systemic scleroderma is a rare, slowly progressive rheumatic disease that is thought to be caused by autoantibodies to nuclear proteins such as SS-A (Ro), SS-B (La), Scl-70, and centromere. Systemic scleroderma can be diffuse or limited. The limited form of systemic scleroderma, or CREST (calcinosis, Raynaud's phenomenon, esophageal involvement, sclerodactyly, and telangiesctasias) can be fatal and involves internal organs less often than diffuse scleroderma. Clinical features of systemic scleroderma include swelling and thickening of the fingers and hand with possible involvement of the face, thickening of the skin, involvement of the trunk and arms proximal to the elbows. As systemic scleroderma progresses, clinical features include skin atrophy with possible loss of hair, sebaceous glands, and sweat glands; loss of pliability of the skin; hidebound skin where the skin is tightly drawn and bound to underlying structures; and limited mobility, especially in the fingers. Scientific American Medicine Chapter 6 Section VI pp. 6-8 and Chapter 15 Section V pp. 1-4 (2001).

Idiopathic thrombocytopenic purpura (ITP) is an autoimmune disorder which is characterized by rapid destruction of the platelets. It is thought that autoantibodies to proteins on platelets are formed and bind to the platelets that are subsequently removed by the reticuloendothelial system. The autoantibodies are frequently directed against the platelet glycoprotein (GP) IIb-IIIa receptor complex. Another target for autoantibodies in ITP is the GPIb receptor complex. Some clinical features of ITP include: presence of petechiae in the lower extremities, mild clinical bleeding consisting of purpura, epistaxis, gingival bleeding, menorrhagia, unpalpable spleen, and in case of several thrombocytopenia, blood blisters in the mouth. Scientific American Medicine Chapter 5 Section XIII pp. 2 (2001).

Polymyositis is a rheumatic disease which involves weakening of primarily skeletal muscle. Polymyositis is thought to be caused by autoantibodies to nuclear proteins such as Jo-1, histadyl-tRNA synthetase, threonyl-tRNA synthetase, PM-1, and Mi-2. Clinical features of polymyositis are weakening of proximal muscles and can also include possible pulmonary involvement such as aspiration pneumonia, interstitial lung disease; soft tissue calcification (seen most commonly in children); and association with another rheumatic disease such as Raynaud phenomenon. Scientific American Medicine Chapter 6 Section VI pp. 6-8 and Chapter 15 Section VI pp. 1-4 (2001).

Systemic lupus erythematosus (SLE) is an autoimmune disease characterized by the production of antibodies to a number of nuclear antigens, including double-stranded DNA (dsDNA). In addition, anti-P₂GPI antibodies can also be found in individuals with SLE. Autoantibodies that react with DNA are believed to play a role in the pathology of SLE and are closely associated with lupus nephritis. See, for example, Morimoto et al. (1982) J. Immunol. 139:1960-1965; Foster et al. (1993) Lab. Invest. 69:494-507; ter Borg et al. (1990) Arthritis Rheum. 33:634-643; and Bootsma et al. (1995) Lancet 345:1595-1599. Other clinical symptoms associated with SLE include malar rash, discoid rash, butterfly rash, photosensitivity, oral ulcers, arthritis, serositis (pleuritis and/or pericarditis), renal disorders (e.g., proteinuria), neurological disorders (e.g., seizures or psychosis), hematological disorders (e.g., hemolytic anemia, leukopenia, lymphopenia, thrombocytopenia), and immunological disorders (e.g., positive lupus erythematosus cell preparation, anti-DNA antibody to native DNA in abnormal titer, anti-SM nuclear antigen antibodies). Coutran et al. Pathologic Basis of Disease Fourth Ed. (1989).

Several methods have been proposed for possible treatment of SLE. One method is the use of dsON conjugated with non-immunogenic carriers, also referred to as platforms. Synthetic dsON have been shown to cross-react with anti-dsDNA antibodies (U.S. Pat. No. 5,276,013). For example, a tetrakis conjugate, LJP 249, composed of four dsON attached to a poly(ethylene glycol) valency platform was used to demonstrate tolerance in an immunized mouse model system (Jones et al. (1994) Bioconjugate Chem. 5:390-399). Another conjugate, LJP 394, which is a tetravalent conjugate composed of four dsON attached to a platform, was shown to delay progression of renal disease and extend survival in the BXSB experimental murine lupus nephritis model (Plunkett et al. (1995) Lupus 4:S99; Coutts et al. (1996) Lupus 5:158-159). LJP 394 has also been shown to lower anti-dsDNA antibodies in human patients with SLE (Weisman et al. (1997) J. Rheumatol. 24:314-318).

Other methods which may be used in the treatment of SLE have been described, including methods of reducing levels of circulating antibodies by inducing B cell tolerance, including, but not limited to, U.S. Pat. Nos. 5,276,013; 5,391,785; 5,786,512; 5,726,329; 5,552,391; 5,268,454; 5,606,047; 5,633,395; 5,162,515; 6,022,544; U.S. Ser. No. 08/118,055 (U.S. Pat. No. 6,060,056); U.S. Ser. Nos. 60/088,656 and 60/103,088 (U.S. Ser. No. 09/328,199 and PCT App. No. PCT/US99/13194).

While SLE is widely considered to be an autoimmune disease, the etiology of SLE is still unknown. B cell expression of human complement receptors 1 and 2 (hCR1 and hCR2) is thought to have some association with SLE. In patients with SLE, abnormalities in the expression of hCR1 and hCR2 (CD21) are routinely observed to be about 50% of levels found in non-SLE patients, as measured by flow cytometry with monoclonal antibodies to hCR1 and hCR2. See, for example, Wilson, J. G. et al. Arthritis Rheum. (1986) 29:739; Levy, E. J., Clin. Exp. Immunol. (1992) 90:235; and Marquart, H. V. et al. Clin. Exp. Immunol. (1995) 101:60.

Human CD21, or complement receptor 2 (CR2), is a membrane glycoprotein of approximately 145-150 kDa which is expressed predominantly on mature B lymphocytes. Tedder, T. F., et al. J. Immunol. (1984) 133:678. Human CD21 is a receptor for complement fragments C3d, C3dg, and iC3b as well as for Epstein-Barr virus (EBV). Weis, J. J., et al. Proc. Natl. Acad. Sci. USA (1984) 81:881 and Fingeroth, J. D., et al. Proc. Natl. Acad. Sci. USA (1984) 81:4150. CD21 consists of approximately 15 to 16 extracellular short consensus repeats (SCR) of about 60 to 70 amino acids each, a transmembrane region of about 24 amino acids, and a short cytoplasmic portion of about 34 amino acids. In mice, CR2 and CR1 are produced by alternative splicing from the same gene, unlike in humans where CR2 and CR1 are unique products of different genes.

CD21 forms a noncovalent receptor complex with CD81 and CD 19 that is important in B cell activation. On mature B cells, CD21 transmits costimulatory signals after cross-linking by polymeric C3d. Melchers, F., et al., Nature (1985) 317:264. Short consensus repeats 1 and 2 (SCR1 and/or SCR2) are thought to be portion of CD21 which specifically recognizes C3d. It is believed that binding of complement fragments, for example C3d, to CD21 plays an important role in B cell responses by providing a link between the B cell antigen receptor and its co-receptor, thus making the B cell 100- to 10,000-fold more sensitive to the antigen. Janeway and Travers Immunobiology 3rd edition (1997) 8:43.

Several compositions for binding agents to CD21 have been described which involve complement or B cell surface proteins. One composition involves recombinant fusion proteins which have been designed for binding to complement fragment. See, for example, WO 91/16437. Another composition which has been described involves binding agents to B cell receptors on endothelial cells. These B cell receptors include CD11b, CD11c, CD21, CD23, a 70 to 85 kDa protein or a 115 kDa protein. See, for example, WO 96/12742, WO 96/12741, or EP 0788513. Yet another composition involves mouse monoclonal antibody to human CD21. This mouse monoclonal antibody is capable of inhibiting infection of CD21-expressing cells with Epstein-Barr virus. Furthermore, it is also capable of interfering with delivery of C3dg-coated antigens to follicular dendritic cells and B cells. See, for example, Prodinger et al. (1998) J. Immunol. 161:4604; U.S. Pat. No. 6,291,239 (Prodinger et al.); EP 1001021 (Prodinger et al.); Guthridge et al. (2001) 167:5758. See also WO 01/92295 (Isenman and Clemenza); U.S. Pat. Nos. 5,552,381 (Atkinson) and 5,719,127 (Atkinson et al.); WO 00/67796 (Curd et al.); WO 96/12742 (Bonnefoy and LeCoanet-Henchoz); U.S. Pat. No. 6,238,670 (Fearon and Dempsey); WO 91/16437 (Hebell and Fearon); EP 528926 (Hebell and Fearon).

Other literature describes the involvement of mouse complement receptors in antibody responses. See, for example, Wiersma, E. J. et al. Eur. J Immunol. (1991) 21:2501-2506 and Takahashi, K., et al. J. Immunol. (1997) 159:1557-1569. Yet other literature describe the study of lymphocyte surface receptors which may be involved in B cell activation in a mouse model of SLE. See, for example, Early, G. S. et al. J. Immunol. (1996) 157:3159-3164; and Mihara, M. et al. (2000) J. Clin. Invest. 106:91-101.

While these studies investigated the involvement of B cell activation receptors CD40 and CD152 in an SLE mouse model, there has been limited investigation into the role that CD21 plays in an ongoing autoimmune response.

There is a need for improved methods of suppressing undesired antibody responses. There is also a need for an improved method of treating patients with autoimmune disease (e.g., SLE) and improved methods for delaying development of autoimmune disease (e.g., SLE) in individuals. The invention provided herein fulfills these needs.

All patents, patent applications, and publications cited herein are hereby incorporated by reference in their entirety.

### SUMMARY OF THE INVENTION

This invention provides methods of treating an individual suffering from an antibody-mediated pathology such as autoimmune disease (e.g., systemic lupus erythematosus (SLE), ITP, or thyroiditis) comprising administering to the individual an effective amount of an agent which interferes with C3d binding to CD21 such that a symptom of the antibody-mediated pathology is ameliorated (i.e., at least one symptom is ameliorated and/or delayed).

In another aspect, the invention provides methods for delaying development of a symptom associated with an antibody-mediated pathology such as autoimmune disease (e.g., systemic lupus erythematosus (SLE), ITP, or thyroiditis) in an individual comprising administering to the individual an effective amount of an agent which interferes with C3d binding to CD21 such that development of a symptom of the antibody-mediated pathology is delayed.

The symptom(s) may be any one or more symptom(s) associated with an antibody-mediated pathology.

### BRIEF DESCRIPTION OF THE DRAWINGS

FIG. 1 depicts IgG anti-oligonucleotide (ON) levels in mice 14 days after priming and 7 days after boosting plus treatment with either PBS (control animals) or soluble CD21 (sCD21, experimental animals).

FIG. 2 depicts IgG anti-oligonucleotide (ON) levels in mice 14 days after boosting plus treatment with either PBS (control animals) or anti-CD21 monoclonal antibody 7G6 (mAb; experimental animals).

FIG. 3 depicts the mean levels of IgM and IgG anti-Galα1-3Gal disaccharide (i.e., digal) in control (PBS) and anti-CD21 (mAb 7G6) treated mice (±1 standard deviation or s.d.).

FIG. 4 is a Kaplan-Meier plot that shows the survival results of NZB×NZW (F1) mice treated with cyclophosphamide, rat anti-mouse 7G6, or both.

### DETAILED DESCRIPTION OF THE INVENTION

We have discovered that inhibiting the interaction of CD21 with C3d suppresses antibody production in antibody-mediated pathologies using art-accepted models for various antibody-mediated pathologies. Antibody-mediated pathologies include, but are not limited to, autoimmune disease, xenotransplantation, and graft-versus-host disease. Based on experimental results, we have found that inhibiting, or interfering with the interaction of CD21 with C3d using agents including but not limited to anti-CD21 monoclonal antibodies suppresses levels of antibodies (e.g., anti-dsDNA antibodies in autoimmune disease such as SLE) in antibody-mediated pathologies.

Accordingly, the invention provides methods of using agents which inhibit CD21 interaction with C3d, presumably to suppress an undesired response in antibody-mediated pathologies for the treatment of antibody-mediated pathologies such as autoimmune disease, for example, SLE. Further, the invention provides for methods of delaying development of antibody-mediated pathologies (e.g., SLE) by administrating to an individual an effective amount of one or more agents which inhibit CD21 interaction with C3d, presumably to suppress antibody production by B cells such that development of a symptom(s) of the antibody-mediated pathology is delayed.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991) and Short Protocols in Molecular Biology (Wiley and Sons, 1999).

Definitions

An “antibody” (interchangeably used in plural form) is an immunoglobulin molecule capable of specific binding to a target, such as a carbohydrate, polynucleotide or polypeptide, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity. An antibody includes an antibody of any class, such as IgG, IgA, or IgM, and the antibody need not be of any particular class.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, human monoclonal antibodies, chimeric (e.g., humanized) monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site (e.g., complementarity determining region or CDR) that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs which are altered with respect to the original antibody.

As used herein, “antibody-mediated pathology” or “antibody-mediated disease” refers to an immune response disorder in which one or more pathologies (including a symptom) are associated with, and more particularly caused by (directly or indirectly) inappropriate and/or undesired production of antibodies. Because an immune response disorder is context dependent, for purposes of this invention, an “antibody-mediated pathology” can encompass autoimmune disease and can also encompass transplantation rejection (especially xenograft rejection and graft-versus-host disease), in which an immune response is inappropriate with respect to attempting to maintain the foreign transplanted tissue, and Rh-based rejection in pregnancy. Antibody-mediated pathology can also encompass pathologies associated with inappropriate production of antibodies in gene therapy. Antibody-mediated pathology can also encompass unwanted or undesirable immune response to a therapeutic agent, such as a therapeutic protein. Examples of such proteins include interferon and heparin (which can give rise to heparin-induced thrombocytopenia). See, for example, Perini (2001) Eur. Cytokine Netw. 12:56-61; Amiral et al. (1998) Platelets 9:77-91.

“Antibody-mediated autoimmune pathology”, “antibody-mediated autoimmune disorder”, or “autoimmune disorder”, as used interchangeably herein, is an immune response in which an abnormal amount of antibodies directed to self-antigens are produced. Antibody-mediated autoimmune pathologies include, but are not limited to, autoimmune disorders such as systemic lupus erythematosus (SLE), antibody-mediated thrombosis, thrombocytopenia (e.g., ITP), anti-phospholipid syndrome (APS), thyroiditis, systemic scleroderma, polymyositis, and myasthenia gravis.

As used herein, “autoimmune” refers to an immune response directed at self-antigens.

As used herein, “autoantibodies” refers to antibodies which are directed to self-antigens. The self-antigens may include but are not limited to nucleic acid (e.g., double-stranded DNA or RNA, single-stranded DNA or RNA, or any combination thereof), nuclear proteins (e.g., SS-A (Ro), SS-B (La), Scl-70, centromere, Jo-1, histadyl-tRNA synthetase, threonyl-tRNA synthetase, PM-1, Mi-2, histones, and chromatin), cellular receptors (e.g., acetylcholine receptor, thyroid-stimulating hormone receptor), cellular proteins (e.g., cardiolipin or β2GP1), RNA protein complexes (e.g., RNP and Sm), erythocytes, and platelet glycoprotein (GP) receptor complexes (e.g., IIb-IIIa and Ib).

As used herein, the term “agent” means a biological or chemical compound such as a simple or complex organic or inorganic molecule, a peptide, a protein, an oligonucleotide, an antibody, an antibody derivative, or antibody fragment. Various compounds can be synthesized, for example oligomers, such as oligopeptides and oligonucleotides, and synthetic organic compounds based on various core structures, and these are also included in the term “agent”. Also included in the term “agent” are antibodies which are generated in animals or synthesized recombinantly or by phage display. In addition, various natural sources can provide compounds for screening, such as plant or animal extracts, and the like. Compounds can be tested and/or used singly or in combination with one another.

An agent that “inhibits or suppresses CD21/C3d mediated B cell activation” is an agent that reduces the extent of CD21/C3d mediated B cell activation mediated by interaction with C3d (i.e., the extent of CD21/C3d mediated B cell activation in the presence of agent and C3d is reduced when compared to the extent of CD21/C3d mediated B cell activation in the presence of C3d without presence of agent). The inhibition of B cell activation can be a partial reduction in activity of B cells such as reduction in the production of antibodies. The inhibition or suppression of CD21/C3d mediated B cell activation may be partial or total. Methods of indicating CD21/C3d mediated B cell activation are known in the art and are described herein. It is understood that “B cell activation” includes the activation of resting B cells, activation of non-antibody-secreting B cells, and sustained and/or enhancement of activation state of B cells which are already activated (e.g., plasma cells). Examples of agents which inhibit CD21/C3d mediated B cell activation include, but are not limited to, antibodies that inhibit CD21 interaction with C3d (can be directed to regions of CD21 to which C3d naturally binds), competitive inhibitors (can bind to C3 and compete for binding to CD21), soluble proteins, fusion proteins, recombinant proteins, and small molecules. For purposes of the methods of this invention, agents described herein inhibit or suppress CD21/C3d mediated B cell activation.

An agent which “interferes with C3d binding to CD21” or “suppresses binding of C3d to CD21” is an agent that reduces the extent of interaction between C3d ligand and CD21 as compared with otherwise same conditions without the agent. Methods for determining C3d binding to CD21 are disclosed herein. An agent which “interferes with C3d binding to CD21” reduces the levels of antibodies such as anti-dsDNA antibodies when appropriately administered.

As used herein, when the terms “inhibit”, “suppress”, “block”, and “interfere” are used to refer to the context of the interaction between CD21 and C3d, these terms mean that the interaction between CD21 and C3d is restricted to completely blocked due to the administration of an agent which hinders the interaction between CD21 and C3d from occurring. To “inhibit” or “suppress” CD21/C3d mediated antibody production means to reduce (which can include elimination) such antibody production.

“Soluble CD21 ” or “sCD21 ” are used interchangeably herein and refers to a soluble (i.e., non-membrane bound) form of CD21. The CD21 can be produced recombinantly or isolated from cells (e.g., CD21 shedding). Soluble CD21 can also be in a variety of lengths, truncated or a fragment of full length CD21 such that it is capable of binding to C3d.

As used herein, “C3d” refers to a complement fragment that is generated as part of the complement pathway. As is well known in the art, C3 is abundant in the plasma. As part of the classical complement pathway, C3 is converted to C3a and C3b by C3 convertase. iC3b is a derivative of C3b and can be further converted to C3dg, for example, when bound to a pathogen as part of an opsonization. As part of the alternative complement pathway, C3b is produced at a significant rate from C3 by spontaneous cleavage (sometimes known as “tickover”). C3b can interact with factor I, a serine protease that circulates in active form and cleaves C3b first into iC3b and then further to C3dg. C3dg can be further degraded to yield C3d. Both C3d and C3dg are capable of binding to CD21. It is understood that C3d and C3dg may be used interchangeably herein. Reference to C3d is understood to also include C3dg and iC3b. iC3b includes the amino acid sequence of C3dg and C3d and binds to CD21 with similar affinity. iC3b is cleaved by proteases to yield C3dg. C3dg has several additional amino acids at the carboxy terminal end which are cleaved by cellular proteases to yield C3d. The structure of C3d is well known in the art (see, for example, Nagar, B. et al. Science 1998. 280, 1277-1281). C3d can be made recombinantly (for example, using published sequences from sources such as Genbank) or obtained in purified form from commercial sources (Calbiochem-Novabiochem; San Diego, Calif., Catalog #204870).

As used herein, “treatment” is an approach for obtaining beneficial or desired results including and preferably clinical results. For purposes of this invention, beneficial or desired clinical results include, but are not limited to, one or more of the following: lowered levels of antibody (for example, anti-double stranded DNA antibody) production (including production levels and/or circulating levels), alleviation of one or more symptoms associated with antibody-mediated pathologies (e.g., autoimmune disease such as SLE, thyroiditis, xenograft rejection, myasthenia gravis, APS, systemic scleroderma, ITP, and polymyositis) listed below, diminishment of extent of an antibody-mediated pathology, stabilized (i.e., not worsening) state of an antibody-mediated pathology, preventing occurrence or recurrence of an antibody-mediated pathology, delaying the development of an antibody-mediated pathology, delay or slowing of an antibody-mediated pathology, amelioration of an antibody-mediated pathology, remission (whether partial or total), reduction of incidence of an antibody-mediated pathology and/or symptoms associated with an antibody-mediated pathology.

Treatment of SLE includes any aspect of SLE, including, but not limited to, immunological disorders (e.g., positive lupus erythematosus cell preparation, anti-DNA antibody to native DNA in abnormal titer, anti-SM nuclear antigen antibodies, anti-β₂GPI antibodies), rashes, photosensitivity, oral ulcers, arthritis, serositis (pleuritis and/or pericarditis), renal disorders (e.g., proteinuria), neurological disorders (e.g., seizures or psychosis), hematological disorders (e.g., hemolytic anemia, leukopenia, lymphopenia, thrombocytopenia, secondary thrombocytopenic purpura), or lupus nephritis, which is a chronic inflammatory kidney disease. During lupus nephritis,“ flares” may occur. “Flares” refer to an increase in activity, generally inflammatory activity. If the activity is in the kidneys, then the flare is referred to as a “renal flare”. “Renal flares” can be identified by evaluating factors including, but not limited to, proteinuria levels, hematuria levels, and serum creatinine levels. The “treatment” of lupus nephritis may be administered when no symptoms of lupus nephritis are present, and such treatment (as the definition of “treatment” indicates) reduces the incidence of flares. Also encompassed by “treatment of lupus” or “treatment of SLE” is a reduction of pathological consequences of any aspect of lupus, such as lupus nephritis.

Treatment of thyroiditis includes any aspect of thyroiditis including, but not limited to, excessive infiltration with chronic inflammatory cells, follicular rupture, eosinophilia, varying degrees of hyperplasia, fibrosis, painless goiter, and hypothyroidism. Treatment of antibody-mediated pathologies associated with xenotransplantation includes any aspect of xenotransplantation including, but not limited to, reduction or elimination of foreign tissue rejection, lowering of antibody titers to the transplanted tissue, and reduction or elimination of graft-versus-host responses. Treatment of myasthenia gravis includes any aspect of myasthenia gravis including, but not limited to skeletal muscle weakness, fatigability, asymmetric ptosis, diplopia, weak neck extensors, drooping of the head, facial snarl when patient attempts to smile due to weakness of facial and bulbar muscles, nasal or dysarthric and low-volume dysphonic speech, dysphagia which can result in choking or regurgitation, and skeletal muscle weakness which can cause difficulties in walking, climbing stairs, or carrying objects. Treatment of systemic scleroderma includes any aspect of systemic scleroderma including, but not limited to, swelling and thickening of the fingers and hand with possible involvement of the face, thickening of the skin, involvement of the trunk and arms proximal to the elbows, skin atrophy with possible loss of hair, sebaceous glands, and sweat glands; loss of pliability of the skin; hidebound skin where the skin is tightly drawn and bound to underlying structures; and limited mobility, especially in the fingers. Treatment of ITP includes any aspect of ITP including, but not limited to presence of petechiae in the lower extremities, mild clinical bleeding consisting of purpura, epistaxis, gingival bleeding, menorrhagia, unpalpable spleen, and in case of several thrombocytopenia, blood blisters in the mouth. Treatment of polyrnyositis includes any aspect of polymyositis including, but not limited to, weakening of primarily skeletal muscle, weakening of proximal muscles, aspiration pneumonia, interstitial lung disease, soft tissue calcification, and Raynaud phenomenon. Treatment of APS (which can also include antibody-mediated thrombosis) includes any aspect of APS including, but not limited to, arterial occlusion, extremity gangrene, stroke, myocardial infarct, other visceral infarct, venous occlusion, peripheral venous occlusion, visceral venous occlusion (e.g., Budd-Chiari syndrome, portal vein occlusion), recurrent fetal loss, thrombocytopenia, Coombs'-positive hemolytic anemia, livedo reticularis, neurological abnormalities (e.g., chorea, transient ischemic attacks), valvular heart disease, and sudden multisystem occlusion.

“Palliating” an antibody-mediated pathology or one or more symptoms of an antibody-mediated pathology means lessening the extent and/or time course of undesirable clinical manifestations of an antibody-mediated pathology in an individual or population of individuals treated with an agent which interferes with CD21/C3d interaction in accordance with the invention.

“Reducing severity of a symptom” or “ameliorating a symptom” of an antibody-mediated pathology means a lessening or improvement of one or more symptoms of an antibody-mediated pathology as compared to not administering an agent which interferes with CD21/C3d interaction. “Reducing severity” also includes shortening or reduction in duration of a symptom. Symptoms of an antibody-mediated pathology such as autoimmune diseases (e.g., SLE), APS, ITP, thyroiditis, xenograft rejection, graft-versus-host, systemic scleroderma, myasthenia gravis, and polymyositis are described supra and can include survival (increased overall survival time as an ameliorated symptom).

As used herein, “delaying” development of an antibody-mediated pathology means to defer, hinder, slow, retard, stabilize, and/or postpone development of the antibody-mediated pathology. This delay can be of varying lengths of time, depending on the history of the antibody-mediated pathology and/or individual being treated. As is evident to one skilled in the art, a sufficient or significant delay can, in effect, encompass prevention, in that the individual does not develop the antibody-mediated pathology. A method that “delays” development of an antibody-mediated pathology is a method that reduces probability of development of the pathologies or symptoms associated with antibody-mediated pathologies in a given time frame and/or reduces extent of the disease in a given time frame, when compared to not using the method. Such comparisons are typically, but not necessarily, based on clinical studies, using a statistically significant number of subjects. For example, a clincician may decide to employ the methods of the invention in an “at risk” individual based on a belief (with or without a basis in a clinical study) that such treatment may lower the risk of the individual with respect to one or more symptoms of the antibody-mediated pathology.

“Development” of an antibody-mediated pathology means the onset and or progression of an antibody-mediated pathology (e.g., autoimmune disease) within an individual. Development of an antibody-mediated, pathology, including autoimmune disease development, can be detectable using standard clinical techniques as described herein. However, development also refers to disease progression that may be initially undetectable. For purposes of this invention, progression refers to the biological course of the disease state, including, for example, the generation of antibodies, including autoantibodies. “Development” includes occurrence, recurrence, and onset. As used herein “onset” or “occurrence” of an antibody-mediated pathology includes initial onset and and/or recurrence.

As used herein, an individual “at risk” is an individual who is considered more likely to develop an antibody-mediated pathology. An individual “at risk” may or may not have detectable disease, and may or may not have displayed detectable disease prior to the treatment methods described herein. “At risk” denotes that an individual has one or more so-called risk factors. An individual having one or more of these risk factors has a higher probability of developing one or more autoimmune disease(s) than an individual without these risk factor(s). These risk factors can include, but are not limited to, history of family members developing one or more antibody-mediated pathologies, history of previous disease, age, sex, race, diet, presence of precursor disease, genetic (i.e., hereditary) considerations, environmental exposure, history of developing malar rashes and butterfly rashes in the case of SLE, or history of recurrent fetal loss in the case of APS. Another example of an “at risk” idividual is one who is or will be receiving a therapeutic agent or therapy which could cause an unwanted antibody response. An “at risk” individual is an example of a suitable individual for receiving the methods of the invention.

An “epitope” is a term well-understood in the art and means any chemical moiety which exhibits specific binding to an antibody. An “epitope” can also comprise an antigen, which is a moiety or molecule that contains an epitope, and, as such, also specifically binds to antibody.

A “double-stranded DNA epitope” or “dsDNA epitope” is any chemical moiety which exhibits specific binding to an anti-double-stranded DNA antibody and as such includes molecules which comprise such epitope(s).

An epitope that “specifically binds” to an antibody is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target (for example, a region of CD21 to which C3d binds) if it binds with greater affinity, avidity, more readily, and/or with greater duration than it binds to other substances. As is well known in the art, one way of detecting specific binding is by competition assays, as described herein.

An “anti-double-stranded DNA antibody” or “anti-dsDNA antibody” or “double-stranded DNA antibody”, used interchangeably herein, is any antibody which specifically binds to double-stranded DNA (dsDNA). Similarly, an “anti-acetylcholine receptor antibody” or “anti-AchR antibody”, used interchangeably herein, is any antibody which specifically binds to acetylcholine receptor. An “anti-thyroid-stimulating hormone receptor antibody” or “anti-TSH receptor antibody”, used interchangeably herein, is any antibody which specifically binds to thyroid-stimulating hormone receptor. An “anti-galα1-3 gal antibody” is any antibody which bind to α-galactose moieties in xenograft rejection. It is understood that nuclear proteins such as SS-A (Ro), SS-B (La), Sc1-70, centromere, histones, chromatin, Jo-1, histadyl-tRNA synthetase, threonyl-tRNA synthetase, PM-1, and Mi-2 or cellular receptors such as acetylcholine receptor, thyroid-stimulating hormone receptor) or cellular proteins such as cardiolipin can be ‘target’ of an antibody and is referred to herein as “anti-‘target’ antibody”. In addition, antibodies to gene therapy vehicles (e.g., adenovirus, adeno-associated virus, retrovirus, etc.) are encompassed as “anti-“target” antibody”.

As clearly indicated in the definition of “antibody” provided herein, an “anti-double-stranded DNA antibody” or any antibody described herein encompasses any fragment(s) that exhibits this requisite functional (i.e., specific binding to dsDNA) property, such as fragments that contain the variable region, such as Fab fragments, and this principle applies to antibodies described herein. As discussed below, it is understood that specific binding to any anti-double-stranded DNA antibody (or functional fragment) is sufficient.

The term “circulating antibody” mean an antibody which is not bound to its cognate epitope on and/or in a biological sample, i.e., free antibody. For example, the term “circulating autoimmune antibody” means an autoimmune antibody which is not bound to its cognate epitope on and/or in a biological sample, i.e., free antibody. In another example, the term “circulating anti-double-stranded (ds) DNA antibody” intends an anti-double-stranded DNA antibody which is not bound to a double-stranded DNA epitope on and/or in a biological sample, i.e., free antibody.

As used herein, the term “immunogen” means a chemical entity that elicits a humoral immune response when injected into an animal. Immunogens have both B cell epitopes and T cell epitopes.

A “T cell epitope” means a component or portion thereof for which a T cell has an antigen-specific specific binding site, the result of binding to which activates the T cell. Where an embodiment of the invention is described as “lacking” a T cell epitope, this is taken to mean that a T cell epitope is not detectable using standard assays in the art. For purposes of this invention, an epitope that “lacks” a T cell epitope means that the epitope lacks a T cell epitope which causes T cell activation in the individual(s) to be treated (i.e., who is to receive an epitope-presenting valency platform molecule). It is likely that, for example, an epitope may lack a T cell epitope(s) with respect to an individual, or a group of individuals, while possessing a T cell epitope(s) with respect to other individual(s). Methods for detecting the presence of a T cell epitope are known in the art and include assays which detect T cell proliferation (such as thymidine incorporation). Immunogens that fail to induce statistically significant incorporation of thymidine above background (i.e., generally p less than 0.05 using standard statistically methods) are generally considered to lack T cell epitopes, although it will be appreciated that the quantitative amount of thymidine incorporation may vary, depending on the immunogen being tested. Generally, a stimulation index below about 2-3, more preferably less than about 1, indicates lack of T cell epitopes. The presence of T cell epitopes can also be determined by measuring secretion of T cell-derived lymphokines according to standard methods. Location and content of T cell epitopes, if present, can be determined empirically. It is understood that, over time, more sensitive assays may be developed to detect the presence of T cell epitopes, and that specifying the lack of T cell epitopes is dependent on the type of detection system used.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. It is understood that the double stranded polynucleotide sequences described herein also include the modifications described herein. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P-NH2) or a mixed phosphoramidate-phosphodiester oligomer. A phosphorothioate linkage can be used in place of a phosphodiester linkage. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers.

As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

“Substantially homologous” refers to sequence homology wherein at least 50% of the sequences are identical, preferably at least 60%, preferably at least 70%, preferably at least 80%, and more preferably at least 90% nucleotide or amino acid residue identity, when compared and aligned for maximum correspondence, as measured using one of the following sequence comparison algorithms or by visual inspection. Two sequences (amino acid or nucleotide) can be compared over their full-length (e.g., the length of the shorter of the two, if they are of substantially different lengths). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols In Molecular Biology, Greene Publishing and Wiley-Interscience, New York, supra).When using any of the aforementioned algorithms, the default parameters for Window length, gap penalty, etc., are used. A further indication that two nucleic acid sequences or polypeptides are substantially identical is that the first polypeptide (e.g., a polypeptide encoded by the first nucleic acid) is immunologically cross reactive with the second polypeptide (e.g., a polypeptide encoded by the second nucleic acid). Thus, a polypeptide is typically substantially identical to a second polypeptide, for example, where the two peptides differ only by conservative substitutions.

“Naturally occurring” refers to an endogenous chemical moiety, such as a carbohydrate, polynucleotide or polypeptide sequence, i.e., one found in nature. Processing of naturally occurring moieties can occur in one or more steps, and these terms encompass all stages of processing. Conversely, a “non-naturally occurring” moiety refers to all other moieties, e.g., ones which do not occur in nature, such as recombinant polynucleotide sequences and non-naturally occurring carbohydrates.

A polynucleotide is said to “encode” a polypeptide if, in its native state or when manipulated by methods well known to those skilled in the art, it can be transcribed and/or translated to produce the polypeptide or a fragment thereof. For purposes of this invention, and to avoid cumbersome referrals to complementary strands, the anti-sense (or complementary) strand of such a polynucleotide is also said to encode the sequence; that is, a polynucleotide sequence that “encodes” a polypeptide includes both the conventional coding strand and the complementary sequence (or strand).

A “fusion polypeptide” is a polypeptide comprising regions in a different position than occurs in nature. The regions may normally exist in separate proteins and are brought together in the fusion polypeptide, or they may normally exist in the same protein but are placed in a new arrangement in the fusion polypeptide. A fusion polypeptide may also arise from polymeric forms, whether linear or branched, for example, the variable region of anti-CD21 monoclonal antibody fused to a marker which may be used for selection, purification, or visualization purposes.

A “host cell” includes an individual cell or cell culture which can be or has been a recipient for vector(s) or for incorporation of polynucleotides and/or proteins. Host cells include progeny of a single host cell, and the progeny may not necessarily be completely identical (in morphology or in genomic of total DNA complement) to the original parent cell due to natural, accidental, or deliberate mutation. A host cell includes cells transfected in vivo with a polynucleotide(s) of this invention.

“Transformation” or “transfection” refers to the insertion of an exogenous polynucleotide into a host cell, irrespective of the method used for the insertion, for example, lipofection, transduction, infection or electroporation. The exogenous polynucleotide may be maintained as a non-integrated vector, for example, a plasmid, or alternatively, may be integrated into the host cell genome.

An “effective amount” (in the antibody-mediated pathology context; such as autoimmune disease) is an amount sufficient to effect beneficial or desired results including clinical results or delaying the onset of the disease. An effective amount can be administered in one or more administrations. For purposes of this invention, an effective amount of an agent described herein (or a composition comprising a agent) is generally, but not necessarily, an amount sufficient to reduce levels of undesired levels of antibodies, e.g., anti-double-stranded DNA antibodies, circulating antibodies, or antibodies deposited as immune complexes. In terms of treatment, an “effective amount” of agent described herein (or a composition comprising an agent) is an amount sufficient to palliate, ameliorate, stabilize, reverse, slow or delay progression of or prevent antibody-mediated diseases such as autoimmune disease (e.g., systemic lupus erythematosus (SLE)), including the progressive inflammatory degeneration of the kidneys that results from SLE (i.e., lupus nephritis). In terms of treatment of other antibody-mediated diseases such as ITP, thyroiditis, myasthenia gravis, systemic scleroderma, polymyositis, and APS, an “effective amount” of an agent described herein is an amount sufficient to palliate, ameliorate, stabilize, reverse, slow, or delay progression of or prevent one or more symptoms of these antibody-mediated diseases including, but not limited to, thrombocytopenia, blood blisters, excessive infiltration with chronic inflammatory cells, graft-versus-host type rejection of foreign tissue, xenograft rejection, follicular rupture, eosinophilia, varying degrees of hyperplasia, fibrosis, painless goiter, hypothyroidism, skeletal muscle weakness, fatigability, asymmetric ptosis, diplopia, weak neck extensors, drooping of the head, facial snarl when patient attempts to smile due to weakness of facial and bulbar muscles, nasal or dysarthric and low-volume dysphonic speech, dysphagia which can result in choking or regurgitation, skeletal muscle weakness which can cause difficulties in walking, climbing stairs, carrying objects, swelling and thickening of the fingers and hand with possible involvement of the face, thickening of the skin, involvement of the trunk and arms proximal to the elbows, skin atrophy with possible loss of hair, sebaceous glands, and sweat glands; loss of pliability of the skin; hidebound skin where the skin is tightly drawn and bound to underlying structures; limited mobility, especially in the fingers, weakening of primarily skeletal muscle, weakening of proximal muscles, aspiration pneumonia, interstitial lung disease, soft tissue calcification, development of Raynaud phenomenon, arterial occlusion, extremity gangrene, stroke, myocardial infarct, other visceral infarct, venous occlusion, peripheral venous occlusion, visceral venous occlusion (e.g., Budd-Chiari syndrome, portal vein occlusion), recurrent fetal loss, thrombocytopenia, Coombs'-positive hemolytic anemia, livedo reticularis, neurological abnormalities (e.g., chorea, transient ischemic attacks), valvular heart disease, and sudden multisystem occlusion. A symptom can include survival.

An “isolated” or “purified” polypeptide or polynucleotide is one that is substantially free of the materials with which it is associated in nature. By substantially free is meant at least 50%, preferably at least 70%, more preferably at least 80%, even more preferably at least 90% free of the materials with which it is associated in nature.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

“In conjunction with” refers to administration of one treatment modality in addition to another treatment modality, such as administration of an agent described herein in addition to administration of another agent (e.g., CD40, CTLA-4) to the same individual. As another example, one anti-CD21 monoclonal antibody administered with another anti-CD21 monoclonal antibody with different sequences but directed to the same epitopes (e.g., SCRI and/or SCR2). As such, “in conjunction with” refers to administration of one treatment modality before, during or after delivery of the other treatment modality to the individual.

“Receiving treatment” includes initial treatment and/or continuing treatment.

An “individual” is a vertebrate, preferably a mammal, more preferably a human. Mammals include, but are not limited to, farm animals, sport animals, pets, primates, mice and rats.

“Comprising” means including.

As used herein, the singular form “a”, “an”, and “the” includes plural references unless indicated otherwise. For example, “an” antibody includes one or more antibodies and “a symptom” means one or more symptoms.

Methods of the invention

With respect to all methods described herein, reference to compositions such as agents which inhibit CD21 interaction with C3d also include compositions comprising one or more of these substances. These compositions may further comprise suitable excipients, such as pharmaceutically acceptable excipients including buffers, which are well known in the art.

Methods of Treating Antibody-Mediated Pathologies by Inhibiting CD21/C3d Mediated B Cell Activation

The invention provides methods of treating antibody-mediated pathologies (e.g., autoimmune diseases) which are characterized by production of antibodies. The methods entail inhibiting, or suppressing, CD21/C3d interaction which can interfere with CD21-mediated B-cell activation, particularly, CD21/C3d mediated activation which can lead to antibody production, including antibody production and autoantibody production.

The methods of the invention entail administering an agent which inhibits CD21/C3d interaction. Lymphocytes which express CD21, generally B cells, are exposed to an agent that inhibits CD2I/C3d interaction, such that CD21-mediated activation of B cells and antibody production may be inhibited. In some embodiments, naturally-occurring CD21-expressing cells (e.g., B cells) are used. In some embodiments, an agent that inhibits CD21/C3d interaction is administered to an individual in an amount sufficient to inhibit CD21/C3d mediated B cell activation and subsequent production of antibodies, generally such that one or more symptoms are alleviated and/or disease development is delayed.

Any agent which inhibits CD21 activation by interfering with CD21/C3d interaction such that CD21/C3d mediated B cell activation and subsequent production of antibodies are inhibited is suitable. Also suitable are agents which inhibit CD21/C3d interaction such that the levels of antibody production are lowered in comparison with the level of antibody production without the use of such agents. In another embodiment, any agent which inhibits CD21/C3d interaction such that the B cells expressing CD21 are delayed in their development into antibody-secreting plasma cells are suitable. Agents which are suitable for inhibition of CD21/C3d interaction include but are not limited to anti-CD21 antibodies, derivatives or fragments thereof, and competitors for cellular CD21 such as soluble CD21 which binds to C3d. In one embodiment, the antibody (-ies) are directed to SCRI and/or SCR2 regions of CD21. Description of suitable agents and methods for making such agents are disclosed herein. In another embodiment, the agent is a soluble CD21 molecule which can bind to C3d and compete with cellular CD21 for binding to unbound C3d.

It is understood that, while presumably the amelioration and/or delaying of development of a symptom of an antibody-mediated pathology is due to inhibition of B cell activation and/or antibody production, and that administration of such an agent may result in such reduction of antibody production, the precise mechanism need not be known. Any agent which interferes with CD21/C3d interaction such that administration contributes to and/or results in such amelioration and/or delaying of development is suitable for the invention.

CD21/C3d mediated B cell activation can detected any number of way known to a skilled artisan. One method that may be used is to detect VDJ recombination using PCR and gel electrophoresis. Another method which may be used to detect B cell activation is by flow cytometry and markers indicative of B cell activation, including but not limited to CD22, CD23, CD24, CD25, CD28, CD30, CD39, CD69, CD72, CD75, CD76, CD86, CD97, CD125, CD126, CD130, and CD153.

The invention uses agents which inhibit CD21/C3d interaction. Inhibition of this interaction may prevent B cell activation and/or suppress antibody production. Preferably, antibody production is inhibited, suppressed, or lowered. Accordingly, levels of antibodies in an individual receiving treatment may be lowered. This method is useful in treating antibody-mediated pathologies such as autoimmune diseases (e.g., SLE), in an individual by administering to the individual an agent which inhibits CD21 interaction with C3d. This method is also useful for delaying development of antibody-mediated pathologies such as autoimmune diseases (e.g., SLE, ITP, or thyroiditis) in an individual. Occurrences of rashes (e.g., butterfly rash) without any development of autoantibodies, fatigue, recurrent pregnancy losses, difficulty with speaking or smiling may be an indication that development of autoantibodies are to follow. The agents described herein can be administered to an individual who has experienced rashes or other initial symptoms associated with SLE prior to development of autoantibodies. The methods provided can also be used to prevent recurrences of SLE or delay indefinitely symptoms associated with SLE.

Several methods may be employed to assess the inhibition of CD21/C3d interaction. These methods are applicable to a variety of agents (e.g., anti-CD21 antibody or soluble CD21) which inhibit CD21/C3d interaction. One method to assess the inhibition of CD21/C3d interaction is to determine binding of an anti-CD21 monoclonal antibody to B cells or other cells expressing CD21 (e.g., from an individual who has received treatment with that antibody). Binding of antibody to CD21 on cells may be determined directly using a secondary antibody that binds to anti-CD21. The secondary antibody may be conjugated to an enzyme (e.g., horseradish peroxidase) or fluorochrome such as fluorescein. It is understood that the secondary antibody should be directed against the animal species of the anti-CD21 antibody. For example, if the agent is a murine anti-CD21 monoclonal antibody, then a secondary anti-mouse IgG conjugated to a visualization marker may be used for, assessing binding. Alternatively, the levels of CD21 on B cells or other cells expressing CD21 from an individual who has received treatment may be determined by measuring the expression of CD21 using an unrelated anti-CD21 antibody conjugated to an enzyme or fluorochrome.

Another method which may be used to assess inhibition of CD21/C3d interaction is to employ ELISA plates coated with C3d, contact a biological sample, for example, B cells or other cells expressing CD21, with the ELISA plate and count the number of cell bound to the ELISA plate. In another alternative, enzymes expressed by live cells be assessed by colorimetric assay (e.g., MTT dye) on the ELISA plate.

Another method which may be employed is coating erythrocytes, for example, sheep red blood cells (SRBC), with complement fragments via the classical pathway of complement activation using anti-erythrocyte antibody and complement. Complement-decorated SRBC may be obtained from the Complement Lab at National Jewish Hospital (Denver, Colo.). Complement-coated SRBC combined with CD21-expressing cells in the absence of anti-CD21 antibodies form “rosettes”. In contrast, complement-coated SRBC combined with CD21-expressing cells in the presence of anti-CD21 antibodies do not form “rosettes” because the binding of the anti-CD21 antibody to CD21 inhibits the binding of CD21 to the SRBC. This resetting technique may also be used to assess the ability of soluble CD21 to inhibit CD21/C3d interaction. An alternative method which may be employed is to use yeast particles incubated in serum to fix complement by activation of the alternative complement pathway. The yeast particles can then be contacted with CD21-expressing cells and monitored for rosette formation. An alternative method that may be employed is to coat fluorescent latex beads (Molecular Probes, Eugene, Oreg.) with complement fragments either by activation of the alternative pathway or by direct conjugation of purified complement fragments. Binding of beads to CD21-expressing cells can be determined by microscopy or by flow cytometry.

Another method which may be used to assess inhibition of CD21/C3d interaction is to obtain a biological sample, e.g., blood, from each individual (at the different incremental dosages) and if desired, separate the T cells from the B cells by subjecting whole blood to sheep red blood cells (SRBC). T cells will preferentially bind to SRBC and T cell-SRBC groups will have a higher density than the B cells. Density centrifugation can separate the T cell-SRBC groups away from the B cells. The B cells can then be contacted with C3d-coated plates in a standard ELISA and if the level of anti-CD21 antibodies (or alternatively, soluble CD21) is sufficient to block all C3d binding regions on the B cells, then little or none of the B cells will bind to the C3d coated plates.

Another method which may be used to assess inhibition of CD21/C3d interaction is utilize anti-CD21 antibodies which have been conjugated to a detectable marker, for example, FITC. A biological sample may be obtained from an individual to whom anti-CD21 antibody conjugated to a detectable marker has been administered. Flow cytometry may used to visualize binding to B cells by monitoring the detectable marker in the appropriate channel (e.g., FLI for FITC) and combining a second antibody (e.g., CD19) to confirm the anti-CD21 antibody has bound to B cells. Once the B cells have been confirmed to have anti-CD21 antibodies bound to them, they can be combined with complement-coated SRBC and monitoring for appearance of “rosettes” as described above.

Another method to assess inhibition of CD21/C3d interaction by agents which inhibit CD21/C3d interaction (e.g., soluble CD21 or anti-CD21 antibodies) is competition assay (e.g., Farr assay). Other methods for assessing inhibition of CD21/C3d interaction by agents which inhibit CD21/C3d interaction (e.g., soluble CD21 or anti-CD21 antibodies) can be used and are known to one of average skill in the art.

Several mouse models of antibody-mediated pathologies such as autoimmune diseases (e.g., SLE) can be used to test agents (e.g., anti-CD21 antibodies or sCD21) which inhibit CD21/C3d interaction to suppress antibody responses in an antibody-mediated pathology. One model is a spontaneous lupus model which utilizes female New Zealand Black×New Zealand White (NZB×NZW) F1 mice. Another model which may be utilized is the MRL 1pr/1pr spontaneous lupus model. MRL 1pr/1pr mice develop symptoms which are similar to human with SLE. These symptoms include but are not limited to high titer anti-dsDNA antibodies, hypocomplementemia, lymphadenopathy, and fatal immune complex-mediated glomerulonephritis.

Another animal model which may be used to assess treatment efficacy is an induced model of anti-DNA antibody production. In this model, oligonucleotide-keyhole limpet hemocyanin (ON-KLH) is administered to mice in an amount effective to induce the formation of anti-dsDNA antibodies. Example 1 discloses how to use ON-KLH to induce anti-ON antibodies in a mouse model. The ON-KLH is made by coupling KLH to a 20-mer double stranded oligonucleotide consisting of (CA)₁₀-(TG)₁₀ and administered in any number of ways described above. Methods of administration include but are not limited to injection (e.g., intraperitoneally, intravenously, subcutaneously, intramuscularly, etc.) The mice may then be allowed to develop a anti-ds ON antibodies and then one or more agents which inhibit the interaction of CD21 with C3d, for example anti-CD21 antibody or soluble CD21 protein, may be administered to the mice. Levels of anti-dsDNA antibodies may be monitored at any time during the course of the experiment, including but not limited to one or more timepoints prior to administration of one or more agent(s) which interfere with the interaction of CD21 with C3d, and at one or more timepoints subsequent to the administration of one or more agent(s) which interfere with the interaction of CD21 with C3d.

Other methods of assessing efficacy of treatment are discussed herein. Other suitable, art-accepted mouse models for various antibody-mediated pathologies are described in the Examples.

Agents for Inhibiting CD21 Interaction with C3d

Methods of the invention entail using agents which interact with CD21 in a manner that inhibits CD21 interaction with complement fragments iC3b, C3d and C3dg. It is understood that C3d and C3dg may be used interchangeably herein. Reference to C3d is understood to also include C3dg and iC3b. iC3b includes the amino acid sequence of C3dg and C3d and binds to CD21 with similar affinity. iC3b is cleaved by proteases to yield C3dg. C3dg has several additional amino acids at the carboxy terminal end which are cleaved by cellular proteases to yield C3d.

Accordingly, agents which are contemplated by the invention include but are not limited to anti-CD21 antibodies, fusion proteins, soluble proteins such as soluble CD21, and recombinant proteins.

Accordingly, one example of an agent which interferes with CD21/C3d interaction that may be utilized is soluble CD21 (sCD21) protein which binds to C3d ligand and competes with cellular CD21 for binding to unbound C3d. Preferably a majority of C3d is bound by sCD21 such that the interaction between cellular CD21 (i.e., CD21 which is attached to cell surface of cells such as B cells and T cells) and unbound C3d is inhibited or suppressed and antibody production is reduced or inhibited. sCD21 can be obtained by following procedures disclosed in Hebell, et al. (1991) Science 254:102 or WO 91/16437.

In some embodiments, the agent can be an anti-CD21 antibody, such as a human or humanized antibody. One example of anti-CD21 antibodies which may be used are antibodies which bind to regions on CD21 to which C3d naturally binds. Short consensus regions 1 and 2 are examples of such regions. In one embodiment, the agent is a mouse anti-human antibody which binds to short consensus region 1 (SCR1) and SCR2 of human CD21. In another embodiment, the agent is a humanized antibody which binds to short consensus region 1 (SCR1) and short consensus region 2 (SCR2) of human CD21. In yet another embodiment, the agent is a human antibody which binds to short consensus region 1 (SCR1) and SCR2 of human CD21. In yet another embodiment, the agent is an antibody which binds to SCR1 or a portion thereof. In another embodiment, the agent is an antibody which binds to SCR2 or a portion thereof.

Antibodies can encompass monoclonal antibodies, polyclonal antibodies, antibody fragments (e.g., Fab, Fab′, F(ab′)₂, Fv, Fc, etc.), chimeric antibodies, single chain (ScFv), mutants thereof, fusion proteins comprising an antibody portion, humanized antibodies, and any other modified configuration of the immunoglobulin molecule that comprises an antigen recognition site of the required specificity. The antibodies may be murine, rat, human, or any other origin. For purposes of this invention, the antibody reacts with human CD21 in a manner that inhibits human CD21 interaction with C3d and may suppress CD21/C3d mediated B cell activation and subsequent antibody production. In one embodiment, the antibody is a mouse or rat antibody which recognizes one or more epitopes on human CD21 to which C3d bind. The epitope(s) can be continuous or discontinuous. Examples of epitopes to which an antibody may be directed include but are not limited to short consensus regions 1 and 2 (SCR1 and/or SCR2). If desired, antibodies which bind to SCR1 and/or SCR2, for example rat monoclonal antibody 7G6, which is directed against mouse CD21 may be obtained from Kinoshita, et al. (1988) J. Immunol. 140:3066 and used in suitable, art-accepted mouse models of disease. In another aspect, antibodies (e.g., human, humanized, mouse, chimeric) which can inhibit CD21 interaction with C3d (e.g., specific for SCRI and/or SCR2 epitopes of CD21) may be made by using immunogens which express CD21. One example of an immunogen is cells with high expression of CD21, e.g., Raji cells which can be obtained from ATCC (accession #CCL-86). Another example of an immunogen which can be used is a soluble CD21 fusion protein which contains the SCR1 and/or SCR2 portion of CD21. The SCRI and/or SCR2 portion of CD21 may be fused with heavy chain IgG, for example, as disclosed in WO 91/16437. Raji cells or CD21 fusion protein may be used alone or in combination with each other as immunogens.

In another aspect, an antibody that binds to human CD21 (in particular, to SCR1 and/or SCR2) that can be used is mouse anti-human monoclonal antibody(mAb) 2B12. mAb 2B12 was generated and characterized as described in Examples 6-11. mAb 2B12 has been shown to disrupt interactions between CD21 and C3d (Example 7). In accordance with the Budapest Treaty, the hybridoma which produces mAb 2B12 has been deposited in the American Type Culture Collection (ATCC), 10801 University Blvd., Manassas Va. 20110-2209 on May 15, 2002, and was accorded a Patent Deposit Designation (Accession Number) of ______. The invention provides this hybridoma or any of its progeny (which may or may not be identical to the deposited hybridoma).

Accordingly, the invention provides any of the following (or compositions, including pharmaceutical compositions, comprising any of the following): (a) antibody 2B 12; (b) antibody produced by the above-referenced hybridoma; (c) a humanized form of antibody 2B12; (d) a humanized form of the antibody produced by the above-referenced hybridoma; (e) an antibody comprising the light chain and/or heavy chain variable regions of antibody 2B12; (f) an antibody comprising the light and/or heavy chain variable regions of an antibody produced by the above-referenced hybridoma; (g) an antibody comprising the light chain and/or heavy chain CDRs of 2B12; (h) an antibody comprising the light chain and/or heavy chain CDRs of an antibody produced by the above-referenced hybridoma. A humanized form of the antibody may or may not have CDRs identical to 2B 12, or antibody produced by the above-referenced hybridoma. Determination of CDR regions is well within the skill of the art. In some embodiments, the invention provides an antibody which comprises at least one CDR that is substantially homologous to at least one CDR, at least two, at least three, at least four, at least 5 CDRs of 2B 12 (or, in some embodiments substantially homologous to all 6 CDRs of 2B 12), or antibody produced by the above-referenced hybridoma. Other embodiments include antibodies which have at least two, three, four, five, or six CDR(s) that are substantially homologous to at least two, three, four, five or six CDRs of 2B 12, or antibody produced by the above-referenced hybridoma. It is understood that, for purposes of this invention, binding specificity and/or overall activity (which may be in terms of suppression of antibody production and/or amelioration of one or more symptoms) is generally retained, although the extent of activity may vary compared to 2B 12 (may be greater or lesser). The invention also provides methods of making any of these antibodies. Methods of making antibodies are known in the art and are described herein.

The route and schedule of immunization of the host animal are generally in keeping with established and conventional techniques for antibody stimulation and production.

It is contemplated that any mammalian subject including humans or antibody producing cells therefrom can be manipulated to serve as the basis for production of mammalian, including human, hybridoma cell lines. Typically, the host animal is inoculated intraperitoneally with an amount of immunogen, e.g., Raji cells or SCR1/SCR2 fusion protein, sufficient to generate an immunogenic response and then boosted with similar amounts of the immunogen. Lymphoid cells, preferably spleen lymphoid cells from the host, are collected a few days after the final boost and a cell suspension is prepared therefrom for use in the fusion.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., (1982) In Vitro, 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-CD21 monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for antigens representative of the type of cells used for immunization.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with Raji cells or SCR1/SCR2 fusion proteins can yield a population of antibodies (e.g., monoclonal antibodies).

If desired, the anti-CD21 antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to SCR1 and/or SCR2. It will be apparent to one of skill in the art that one more polynucleotide changes can be made to the anti-CD21 antibody and still maintain its binding ability to SCR1 and/or SCR2 regions or epitopes of CD21.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; and 6,331,415.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; and 5,866,692.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In an alternative, antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. (2001) Vaccine 19:2756; Lonberg, N. and D. Huszar (1995) Int.Rev.Immunol 13:65; and Pollock, et al.(1999) J Immunol Methods 231:147. Methods for making derivatives of antibodies, e.g., humanized, single chain, etc. are known in the art. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. (1994) 12:433-455.

The antibodies made either by immunization of a host animal or recombinantly should exhibit all of the following characteristics: (a) binds to CD21; (b) binds to one or more epitopes of CD21 to which C3d binds; (c) binds to CD21 to inhibit CD21/C3d-mediated B cell activation (d) binds to CD21 to inhibit CD21/C3d-mediated B cell activation and lower levels of antibody production.

Immunoassays and flow cytometry sorting techniques such as fluorescence activated cell sorting (FACS) can also be employed to isolate antibodies that are specific for CD21 and more preferably, the SCR1 and/or SCR2 epitopes of CD21. For example, ELISA with C3d-coated and soluble CD21-coated plates may be employed to determine which antibodies are specific for C3d binding portion of CD21 (i.e., SCR1 and/or SCR2). Flow cytometry may be used to assess how well the antibody(-ies) bind to CD21-expressing cells, including but not limited to B cells or a cell line such as Raji. In the alternative, antibodies can be screened by combining with a population of B cells and then exposing the B cells to a source of C3d, either in isolated form (e.g. C3d coated plates) or in natural form (e.g., in serum). Flow cytometry and markers indicative of B cell activation, including but not limited to CD22, CD23, CD24, CD25, CD28, CD30, CD39, CD69, CD72, CD75, CD76, CD86, CD97, CD125, CD126, CD130, and CD153 may be used to detect how well the anti-CD21 antibody inhibits B cell activation.

The antibodies cari be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

The antibodies can also be conjugated to a detectable agent. The complex is useful to detect the antigens to which the antibody specifically binds in a sample, using standard immunochemical techniques such as flow cytometry or immunohistochemistry as described by Harlow and Lane (1988) supra. Detectable markers can also be used to ascertain binding specificity for a type of cell (e.g., B cell) by using the detectable marker with another marker which is definitive for B cells (e.g., CD19, CD20, CD22, etc.) and analyzing the staining patterns by FACS. There are many different labels and methods of labeling known to those of ordinary skill in the art. Examples of the types of labels which can be used in the present invention include radioisotopes, enzymes, colloidal metals, fluorescent compounds (e.g., FITC, PE, PECy5, APC, etc.), bioluminescent compounds, and chemiluminescent compounds. Those of ordinary skill in the art will know of other suitable labels for binding to the antibody, or will be able to ascertain such, using routine experimentation. Furthermore, the binding of these labels to the antibody of the invention can be done using standard techniques common to those of ordinary skill in the art.

Administration of Agents

Various formulations of agents such as antibodies or fragments thereof may be used for administration. In some embodiments, agent(s) such as the anti-CD21 antibodies or fragments thereof may be administered neat. In some embodiments, the agents comprise anti-CD21 antibodies or fragments thereof and a pharmaceutically acceptable excipient, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intramuscularly, etc.). Accordingly, these agents are preferably combined with pharmaceutically acceptable vehicles such as saline, Ringer's solution, dextrose solution, and the like. The particular dosage regimen, i.e., dose, timing and repetition, will depend on the particular individual and that individual's medical history. Empirical considerations, such as the half life, generally will contribute to determination of the dosage. Frequency of administration may be determined and adjusted over the course of therapy, and is generally, but not necessarily, based on maintaining reduction of antibody production and/or suppression/amelioration/delay of one or more symptoms. Other appropriate dosing schedules may be as frequent as continuous infusion to daily or 3 doses per week, or one dose per week, or one dose every two to four weeks, or one dose on a monthly or less frequent schedule depending on the individual or the antibody-mediated disease state. Repetitive administrations, normally timed according to B cell turnover rates, may be required to achieve and/or maintain a state of suppression of CD21/C3d interaction to treat antibody-mediated pathologies such as autoimmune diseases (e.g., SLE). Alternatively, sustained continuous release formulations of the agents may be appropriate. Various formulations and devices for achieving sustained release are known in the art.

In an alternative, dosing regime may be adjusted accordingly for an individual who has a family history of an antibody-mediated pathology such as autoimmune diseases (e.g., SLE) to delay development of the antibody-mediated pathology. In such cases, consideration is made to balance side effects resulting from possible toxicity with an effective dosage.

In one embodiment, dosages for agents may be determined empirically in individuals who have been given one or more administration(s) of an agent which inhibits CD21/C3d interaction to treat an antibody-mediated pathology such as autoimmune diseases (e.g., SLE). Individuals are given incremental dosages of an agent which inhibits CD21/C3d interaction, e.g., anti-CD21 antibody. A biological sample, e.g., blood, is obtained from each individual at the different incremental dosages and if desired, the T cells may be separated away from the B cells by subjecting whole blood to sheep red blood cells (SRBC). T cells will preferentially bind to SRBC and T cell-SRBC groups will have a higher density than the B cells. Density centrifugation can separate the T cell-SRBC groups away from the B cells. The B cells can then be contacted with C3d-coated plates in a standard ELISA and if the level of anti-CD21 antibodies is sufficient to block all C3d binding regions on the B cells, then little or none of the B cells will bind to the C3d coated plates. In the alternative, SRBC coated with complement may be used in a rosetting assay, as disclosed supra. In another alternative, loss of CD21 on the surface of cells in a treated individual may also be monitored, for example, by FACS. Loss of CD21 can occur by downregulation of CD21 on the cell surface upon treatment with one or more of the agents disclosed herein or by shedding of CD21. See, for example, Fremeaux-Bacchi, et al. (1999) Immunopharmacology 42:31.

It will be apparent to one of skill in the art that the dosage could vary depending on the individual, the stage of the antibody-mediated pathology and composition of B cells within the individual. Further, an individual who has a greater composition of B cells as an overall percentage of their lymphocytes may require a higher dosage than another individual with a lower percentage of B cells. An individual developing symptoms of SLE, e.g., anti-dsDNA antibodies or anti-β₂GPI antibodies (which can also be found in individuals with APS or antibody-mediated thrombosis), may require a higher dosage of agents which inhibit CD21/C3d interaction than another individual with low or no levels of anti-dsDNA antibodies.

Other formulations include suitable delivery forms known in the art including, but not limited to, carriers such as liposomes. Mahato et al. (1997) Pharm. Res. 14:853-859. Liposomal preparations include, but are not limited to, cytofectins, multilamellar vesicles and unilamellar vesicles.

In some embodiments, more than one agent, such as an antibody, may be present. The agents can be the same or different from each other. Such agents may contain at least one, at least two, at least three, at least four, at least five different antibodies. Anti-CD21 antibody can be admixed with one or more antibodies reactive against B cell surface proteins, including but not limited to CD 19, CD20, CD23, CD28, CD38, CD40, CD45, CD45R, or CD81. In one embodiment, the B cell surface proteins are proteins which are involved in B cell activation or B cell maturation. The B cell maturation may be at any stage of B cell development. In one embodiment, the antibodies are a mixture of antibodies which target activation pathways, for example CD19 and CD81. In other embodiments, the antibodies are a mixture of antibodies which target specific population of B cells during development, for example immature B cells, mature naive B cells, lymphoblasts, memory B cells, or plasma cells. In yet another embodiment, the anti-CD21 antibody is admixed with antibodies reactive with T lymphocyte surface proteins which are involved in helping B cell activation and/or maturation. Examples of such proteins include but are not limited to CD40 ligand, CD152 (CTLA4), and CD28. A mixture of antibodies, as they are often denoted in the art, may be particularly useful in treating a broader range of population of individuals. It is understood that any population of B cells within an individual will contain a mixture of immature, partially mature, and mature B cells undergoing different stages of development. The combination of anti-CD21 antibodies with other anti-lymphocyte proteins may also be useful in being more effective than using only one (or fewer than are contained in the cocktail) antibody(ies).

In some embodiments, the methods entail administering an agent (which may be an antibody) in conjunction with another therapy or treatment modality. In some embodiments, the other therapy is other than a CD21 based therapy (i.e., the additional therapy does not affect the CD21/C3d interaction). Accordingly, the invention provides methods which further comprise administration of another or additional treatment (or therapeutic agent) which can include one or more additional treatments (therapeutic agents). The additional treatment (or therapeutic agent) may be any which is used for an antibody-mediated pathology (including agents or other treatments, such as radiation). For example, an agent may be administered in addition to administration of one or more immunosuppressants, such as corticosteroid cyclophosphamide immunosuppressants to the same individual, which is suitable for lupus. With respect to, for example, immunosuppresant therapy, one or more additional therapeutic agents may be administered, and such combination therapies are known in the art. Administration (including formulations, dosing, etc.) of cyclophosphamide (as well as other immunosuppressants) is known in the art. This combination could reduce the need for other therapeutic agents (in terms of, for example, less frequent administration and/or dosing), which often have negative side effects. Conversely, these conjunctive therapies could also permit higher dosing due to a protective effect of the CD21 based therapy.

Methods of Assessing Efficacy of Treatment

Assessment of treatment efficacy can be performed on several different levels. Assessment may be made by monitoring clinical signs (e.g., symptoms associated with antibody-mediated pathologies, for example SLE, ITP, or thyroiditis), cellular responses (e.g., antibody secretion), or molecular changes within one or more cells (e.g., B cell activation markers). Examples of various measures for treatment of antibody-mediated pathologies have been discussed above.

Detection and measurement of efficacy in treatment of an antibody-mediated pathology such as autoimmune disease are generally based on detection of and measurement of levels of autoimmune responses to self-antigens and/or other symptom(s) associated with autoimmune diseases. Levels of autoimmune responses can be reflected in antibody titers to self-antigens. Preferably, the antibody titers to self-antigens are reduced after treatment with one or more agents of this invention as compared to antibody titers prior to treatment with one or more agents of this invention. In the case of SLE, measurement of anti-double-stranded DNA (anti-dsDNA) antibody, anti-SM nuclear antigen antibodies, anti-β₂GPI antibodies (which can also be found in individuals with APS or antibody-mediated thrombosis), and/or clinical symptoms associated with SLE, which are known in the art may be used as a measure of efficacy. Measurement of anti-dsDNA antibody levels can be accomplished by testing routinely employed in clinical settings (e.g., Farr assay or ELISA). Examples of other SLE-associated symptoms include but are not limited to malar rash, discoid rash, butterfly rash, photosensitivity, oral ulcers, arthritis, serositis (pleuritis and/or pericarditis), renal disorders (e.g., proteinuria), neurological disorders (e.g., seizures or psychosis), hematological disorders (e.g., hemolytic anemia, leukopenia, lymphopenia, thrombocytopenia), and lupus nephritis. Lupus nephritis (kidney glomerulonephritis or kidney inflammation) is characterized by a progressive loss of kidney function culminating in renal failure. Lupus nephritis is characterized by hematuria, decreased urine output, elevated blood urea nitrogen levels, elevated serum creatinine levels, hypertension, and proteinuria. Accordingly, these parameters can be monitored as a means of monitoring kidney degeneration.

In the case of thyroiditis, determination of efficacy of treatment can include, but is not limited to, measurement of levels of antibody titers to thyroid-stimulating hormone receptor and amelioration or palliation of symptoms associated with thyroiditis including, but not limited to, excessive infiltration with chronic inflammatory cells, follicular rupture, eosinophilia, varying degrees of hyperplasia, fibrosis, painless goiter, and hypothyroidism.

In the case of ITP, determination of efficacy of treatment can include, but is not limited to, measurement of levels of platelets, amelioration of clinical bleeding (e.g., purpura, epistaxis, gingival bleeding, or menorrhagia), dissipation of blood blisters, and petechiae of lower extremities.

In the case of xenotransplantation, determination of efficacy of treatment can include, but is not limited to, measurement of levels of antibody titers to the transplanted (i.e., xenogeneic) tissue, reduction or elimination of graft-versus-host responses, and lessening of rejection of transplanted tissue rejection (e.g., reduction or elimination of necrotic tissue) as determined by a skilled artisan.

In the case of APS (which can also include antibody-mediated thrombosis), determination of efficacy of treatment can include, but is not limited to, measurement of levels of antibody titers to anti-phospholipid, cardiolipin, or β₂-GPI. Additionally, non-limiting symptoms associated with APS include arterial occlusion, extremity gangrene, stroke, myocardial infarct, other visceral infarct, venous occlusion, peripheral venous occlusion, visceral venous occlusion (e.g., Budd-Chiari syndrome, portal vein occlusion), recurrent fetal loss, thrombocytopenia, Coombs'-positive hemolytic anemia, livedo reticularis, neurological abnormalities (e.g., chorea, transient ischemic attacks), valvular heart disease, and sudden multisystem occlusion.

In the case of myasthenia gravis, determination of efficacy of treatment can include, but is not limited to, measurement of levels of antibody titers to acetylcholine receptor and amelioration or palliation of symptoms associated with myasthenia gravis including, but not limited to, skeletal muscle weakness which can cause difficulties in walking, climbing stairs, or carrying objects, fatigability, asymmetric ptosis, diplopia, weak neck extensors, drooping of the head, facial snarl when patient attempts to smile due to weakness of facial and bulbar muscles, nasal or dysarthric and low-volume dysphonic speech, and dysphagia which can result in choking or regurgitation.

In the case of systemic scleroderma, determination of efficacy of treatment can include, but is not limited to, measurement of levels of antibody titers to nuclear proteins such as SS-A (Ro), SS-B (La), Sc1-70, and centromere. Additionally, non-limiting symptoms associated with systemic scleroderma include swelling and thickening of the fingers and hand with possible involvement of the face, thickening of the skin, involvement of the trunk and arms proximal to the elbows, skin atrophy with possible loss of hair, sebaceous glands, and sweat glands; loss of pliability of the skin; hidebound skin where the skin is tightly drawn and bound to underlying structures; and limited mobility, especially in the fingers.

In the case of polymyositis, determination of efficacy of treatment can include, but is not limited to, measurement of levels of antibody titers to nuclear proteins such as Jo-1, histadyl-tRNA synthetase, threonyl-tRNA synthetase, PM-1, and Mi-2. Additionally, non-limiting symptoms associated with polymyositis include weakening of primarily skeletal muscle, weakening of proximal muscles, aspiration pneumonia, interstitial lung disease, soft tissue calcification, and Raynaud phenomenon.

In general, measuring appropriate antibody titer (depending on the disease context) is suitable for monitoring disease state as well as appropriate dosages. For purposes of the invention, one or more symptoms is ameliorated (including, where appropriate, incidence and frequency of events) and/or delayed.

Kits

The invention provides kits for carrying out the methods of the invention. Accordingly, a variety of kits are provided in suitable packaging. The kits may be used for any one or more of the uses described herein, and, accordingly, may contain instructions for any one or more of the following uses: treating an antiobody-mediated pathology; delaying development of an antibody-mediated pathology.

The kits of the invention comprise one or more containers comprising any of the agents, for example, an antibody, described herein. Each component (if there is more than one component) can be packaged in separate containers or some components can be combined in one container where cross-reactivity and shelf life permit.

The kits of the invention may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The following examples are provided to illustrate, but not limit, the invention.

### EXAMPLES

### Example 1

### Use of sCD21 to Reduce Levels of Anti-dsDNA Antibodies

C57/B6 mice were primed with model T-dependent antigen, Keyhole Limpet Hemocyanin (KLH) coupled to a 20-mer double stranded oligonucleotide consisting of (CA)₁₀-(TG)₁₀ (ON-KLH )in the ratio of 3.8 mole oligonucleotide/mole KLH. Mice were bled then immunized with 50 μg of alum precipitated ON-KLH i.p. together with 2×10⁹ killed B. Pertussis organisms as additional adjuvant. Fourteen days later, mice were boosted with 10 μg ON-KLH i.p. At the time of boosting, mice received 300 μg soluble CD21 (sCD21; Hebell, et al. (1991) Science 254:102) i.v. plus 300 μg sCD21 i.p. Control mice received PBS. For the next 3 days, mice received 300 μg sCD21 or PBS i.v. daily.

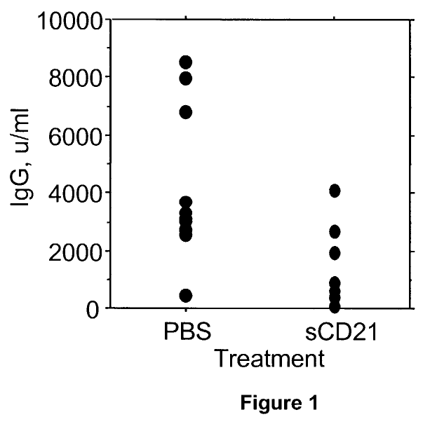

Mice were bled 7 days after boosting and serum IgG levels specific for ON were measured by ELISA against ON conjugated to bovine serum albumin. Concentration of antibody to ON in arbitrary units was determined by comparison with a standard pool of immunized mouse serum. Treatment with sCD21 significantly reduced the level of IgG anti-ON (p=0.02, Mann-Whitney U test) as shown in Table 1 and FIG. 1.

Table 1: IgG anti-ON levels in mice after priming (u/ml, Pre) and 7 days after boosting plus treatment with PBS or sCD21 (u/ml, Post).

TABLE 1

| Mouse # | Treatment | Pre | Post |
|---|---|---|---|
| A1 | PBS | 558 | 7960 |
| A2 | PBS | 229 | 8531 |
| A3 | PBS | 149 | 3014 |
| A4 | PBS | 7 | 448 |
| A5 | PBS | 63 | 2777 |
| B1 | PBS | 51 | 2555 |
| B2 | PBS | 1 | 3717 |
| B3 | PBS | 212 | 3340 |
| B4 | PBS | 323 | 6823 |
| B5 | PBS | 238 | 3137 |
| Mean | PBS | 183 ± 171 | 4230 ± 2626 |
| E1 | sCD21 | 88 | 1929 |
| E4 | sCD21 | 14 | 409 |
| F1 | sCD21 | 2 | 86 |
| F2 | sCD21 | 58 | 2732 |
| F3 | sCD21 | 175 | 909 |
| F4 | sCD21 | 29 | 599 |
| F5 | sCD21 | 617 | 4114 |
| Mean | sCD21 | 141 ± 218 | 1540 ± 1465 |

There was a 64% reduction in the mean levels of antibody in the sCD21 treated group compared to the PBS treated group.

### Example 2

### Use of anti-CD21 Antibody to Reduce Levels of Anti-dsDNA Antibodies

BALB/c mice were primed with a model T-dependent antigen, Keyhole Lymphocyte Hemocyanin (KLH) coupled to a 20-mer double stranded oligonucleotide consisting of (CA)₁₀-(TG)₁₀ (ON-KLH) in the ratio of 3.8 mole oligonucleotide/mole KLH.

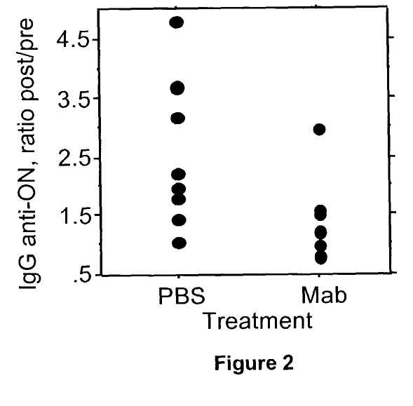

Mice were bled then immunized with 50 μg of alum precipitated ON-KLH i.p together with 2×10⁹ killed B. Pertussis organisms as additional adjuvant. After 10 weeks, mice were bled and treated with PBS or with 200 μg rat monoclonal antibody 7G6 i.v. Rat monoclonal antibody 7G6 (IgG2b anti-mouse CD21 antibody) is described in Kinoshita, et al. (1988) J. Immunol. 140:3066. Twenty four hours later, mice were boosted with 10 μg ON-KLH i.p. IgG antibodies specific for ON were measured 14 days after boosting by ELISA against ON. In this experiment, mice had significant levels of IgG anti-ON prior to boosting, therefore the effect of treatment was determined by comparing the ratio of IgG anti-ON pre and post boost. Treatment with monoclonal antibody significantly reduced the level of IgG anti-ON (p=0.03, Mann-Whitney U test). Data are shown in Table 2 and depicted in FIG. 2.

TABLE 2

| Mouse # | Treatment | u/ml, Pre | u/ml, Post | Ratio, post/pre |
|---|---|---|---|---|
| A2 | PBS | 427 | 1575 | 3.69 |
| A3 | PBS | 480 | 2302 | 4.79 |
| A4 | PBS | 598 | 1898 | 3.17 |
| A5 | PBS | 732 | 1300 | 1.78 |
| B1 | PBS | 3891 | 8566 | 2.20 |
| B2 | PBS | 1274 | 1318 | 1.04 |
| B3 | PBS | 1642 | 3198 | 1.95 |
| B5 | PBS | 801 | 1146 | 1.43 |
| Mean | PBS | 1231 ± 1052 | 2663 ± 2478 | 2.51 ± 1.27 |
| C2 | Mab 7G6 | 4806 | 3695 | 0.77 |
| C3 | Mab 7G6 | 1118 | 3309 | 2.96 |
| C4 | Mab 7G6 | 1911 | 1877 | 0.98 |
| C5 | Mab 7G6 | 2034 | 1628 | 0.80 |
| D1 | Mab 7G6 | 1326 | 1987 | 1.50 |
| D3 | Mab 7G6 | 606 | 729 | 1.20 |
| D4 | Mab 7G6 | 994 | 1567 | 1.58 |
| D5 | Mab 7G6 | 4663 | 5643 | 1.21 |
| Mean | Mab 7G6 | 2182 ± 1643 | 2554 ± 1575 | 1.37 ± 0.7 |

There was a 45% reduction in the ratio of post/pre antibody levels in the antibody treated group compared to the PBS treated group.

### Example 3A

### Use of anti-CD21 Antibody to Reduce Levels of Anti-dsDNA Antibodies in a Spontaneous Model of Lupus

The NZB/WF1 (H-2^{d/z}) mouse strain provides an autoimmune model to study the complex mechanisms controlling the onset of autoimmunity in lupus. The animals are F1 hybrid offspring of NZB/B1NJ females and NZW/LacJ males, and are obtained from the Jackson Laboratory (Bar Harbor, Me.). The mice develop a chronic, inflammatory disease of unknown origin that is strongly influenced by genetic factors. Autoimmunity manifests itself as high levels of circulating anti-nuclear antibodies (including anti double-stranded DNA antibodies); autoantibodies to RNA-protein complexes (e.g., RNP and Sm), ssDNA, histone, chromatin, erythrocytes, and cardiolipin; immune complex formation with deposition in the kidneys leading to proteinuria; and progressive glomerulonephritis. Female animals are used in this study since they have a much higher incidence and severity of disease than the males (average lifespan for females is ˜35 weeks and for males ˜1 year, according to The Jackson Laboratory).

Each study consists of groups of animals at different stages of disease (detailed below), and include:

(1) control group that receives no treatment (saline), (2) control group that receives isotype control antibody, and (3) test group that receives test reagents, either alone or in combination with each other.

Between 4 and 6 months of age, mice are assigned to 4 study groups based on disease stage as monitored by proteinuria. The disease stages are as follows:

pre-disease onset=none.

1+<30 mg/d1 (mild)

2+<100 mg/d1 (severe)

3+>100 mg/d1 (advanced)

Animals are treated with rat IgG2b anti-mouse CD21 antibody (7G6) or a rat IgG2b isotype control (Kinoshita et al. (1990) Int. Immunol. 2:651-659). Alternatively, a soluble CD21-Ig fusion protein or a mouse IgGI control (Hebell, T. et al. (1991) Science 254:102-105) is used. Cyclophosphamide is also used for general immunosuppression and to test for synergy with the antibodies.

Within each group (minimum of 10 mice per group), mice are treated with one of the following regimens:

1. Anti-CD21 500 μg via the tail vein twice weekly (day 3 and day 7).

2. CD21-Ig 500 μg via the tail vein twice weekly (day 3 and day 7).

3. Cyclophosphamide 1 mg intraperitoneally weekly (day 7).

4. Rat IgG2 500 μg via the tail vein twice weekly (day 3 and day 7).

5. Mouse IgG1 500 μg via the tail vein twice weekly (day 3 and day 7).

6. Saline via the tail vein twice weekly (day 3 and day 7).

7. 1 and 3 combined.

8. 2 and 3 combined.

Following treatment, mice are bled weekly for determination of anti-dsDNA and anti-β2GPI antibody titers as well as titers of autoantibodies to RNA-protein complexes (e.g., RNP and Sm), ssDNA, histone, chromatin, erythrocytes, and cardiolipin. Levels of proteinuria are also assessed weekly to monitor progression of autoimmune disease.

Several assays are used for determination of autoimmune disease onset or progression. The following are assays which are used:

1) Anti-dsDNA ELISA. Titers of anti-double-stranded DNA antibodies in animal sera are determined using a solid-phase ELISA.

2) Titers to antibodies for anti-RNP, anti-Sm, anti-ssDNA, anti-histone, anti-chromatin, anti-erthryocyte, and anti-cardiolipin are measured by ELISA.

3) Renal Disease. Proteinuria will be measured using commercially available dipsticks (Ames Uristix; Bayer Diagnostics, Catalog No. 2184, Tarrytown, N.Y.).

4) Anti-β2-glycoprotein I (β2GPI) ELISA. Titers of antibodies to (β2GPI are measured using a solid-phase ELISA.

5) Survival. The rate of survival for the experimental mice is monitored.

### Example 3B

### Animal Studies Using Anti-CD21 Antibody

60 female (NZB)×(NZW) F1 mice were bled at 18 weeks of age, and the titer of serum antibodies to double-stranded-(ds) DNA measured by a standard ELISA assay employing salmon milt DNA (Calbiochem, San Diego, Calif.) as substrate. Mice were randomized into 4 groups of 13 to 15 mice, such that each group contained mice with comparable serum anti-DNA antibody levels. The animals were then started on one of 4 treatment regimens:

A) No treatment

B) Cyclophosphamide administered as a single intraperitoneal injection in sterile phosphate-buffered saline at 40 mg/kg weekly (on day 3).

C) Rat IgG2bK anti-mouse CD21 mAb 7G6 administered as a single intraperitoneal injection in sterile phosphate-buffered saline at 250 μg weekly (on day 7).

D) Intraperitoneal injections of both Cyclophosphamide (day 3) and 7G6 (day 7) at the indicated doses.

Treatments were carried out between 18 and 36 weeks of age only. During this time the mice were analyzed biweekly to determine weight, proteinuria levels, hematocrits and serum anti-dsDNA antibody concentrations. In addition, animals were monitored daily for signs of distress and moribund animals were euthanized.

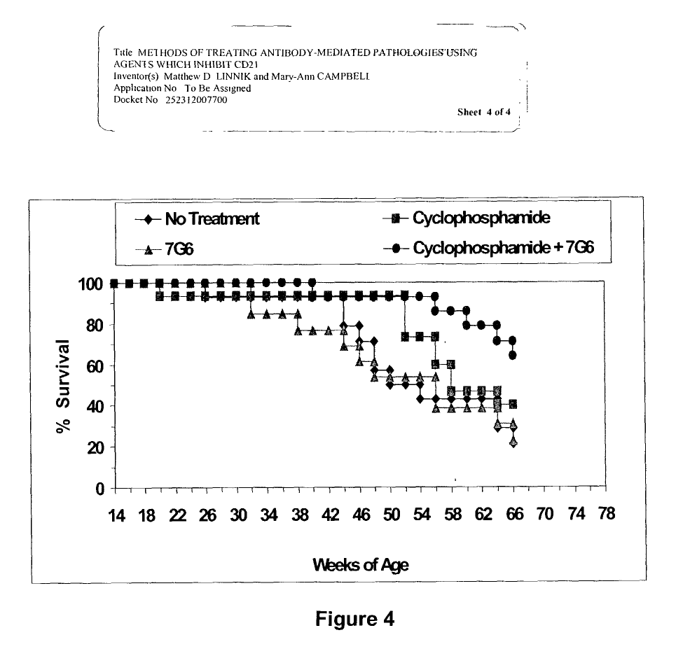

The influence of each treatment on survival is shown in the Kaplan-Meier plot (FIG. 4). When compared with no treatment, injection of anti-CD21 mAb alone did not prolong survival, while the immunosuppressant agent cyclophosphamide did. The most striking affect on prolonging survival was observed in the group of animals receiving both cyclophosphamide and mAb 7G6.

There was no effect of anti-CD21 (7G6) treatment on total anti-dsDNA antibody concentrations (whether administered alone or in combination with cyclophosphamide) when compared to control mice (no treatment) or mice treated with cyclophosphamide alone.

### Example 4

### Suppression of Antibodies to gal α1-3gal in α1,3-Galactosyltransferase Knockout Mice Using Monoclonal anti-CD21 Antibody 7G6

α1,3-galactosyltransferase knockout mice (Thall, et al. J. Biol. Chem. (1995) 270:21437-21440) (GalT KO) are unable to synthesize the Galal-3Gal disaccharide (digal) and as such are similar to humans, apes, and Old World monkeys in that they spontaneously form antibodies that recognize terminal Galα1-3Gal epitopes. Natural anti-Galα1-3Gal antibodies in humans present a significant barrier to xenotransplantation, causing hyperacute rejection of xenografts. Therefore, the GalT KO mouse model is an extremely useful system in which to test potential methods of diminishing natural anti-Galα1-3Gal antibodies (Yang, et al. J. Exp. Med. (1998) 187:1335-1342).

The goal of this experiment was to determine whether treatment of GalT KO mice with monoclonal antibody 7G6, specific for CD21, would prevent the induction of anti- Galα1-3Gal antibodies in response to immunization with rabbit red blood cells, a source of Galα1-3Gal disaccharide.

GalT KO mice were obtained from John Lowe (University of Michigan). Mice (11 weeks old, 10 per group) were treated i.p. with 600 μg 7G6 in phosphate buffered saline (PBS) or PBS alone. One day later mice were immunized with 1×10⁹ rabbit red blood cells i.p. Animals were bled 8 days later and serum samples were assayed for anti-digal-antibodies by ELISA. Immunoassay plates (Costar # 3590) were coated overnight at 4° C. with 100 μl/well digal-bovine serum albumin (BSA-digal) at 5 μg/ml in phosphate buffered saline (PBS). Plates were washed with PBS and remaining protein-binding sites blocked overnight at 4° C. with 250 μl/well 5% non-fat dried milk in PBS. Serum samples were diluted in Hanks Balanced salt solution (HBSS) containing 0.5% BSA (HBSA). Blocked plates were washed with PBS-0.1% Tween 20 and 50 μl of serum sample dilutions added. Plates were incubated for 1 hour at room temperature. Plates were washed with PBS-0.1% Tween 20. Alkaline phosphatase-conjugated goat anti-mouse IgG or IgM (Jackson # 115-055-146 or 115-055-075, 1/1000 in HBSA) was added (100 μl/well) and plates were incubated for 1 hour at room temperature. Plates were washed with PBS-0.1% Tween 20. Phenolpthalein monophosphate (1:26 in distilled water, 100 μl per well) was added and the plates incubated at room temperature. Optical density was read at 550 nm after 10 and 30 minutes incubation on a PowerWave 340 Microplate spectrophotometer.

Serum levels of anti-digal-antibodies in arbitrary units/ml were determined by comparison with a standard curve generated using serum pooled from rabbit red cell-immunized GalT KO mice.

Serum anti-digal antibody levels were analyzed by Students t-test using Statview. IgM and IgG anti-digal antibodies were significantly reduced in 7G6-treated animals relative to placebo treated animals (IgM, p=0.0085, IgG, p=0.0146, Mann-Whitney U test).

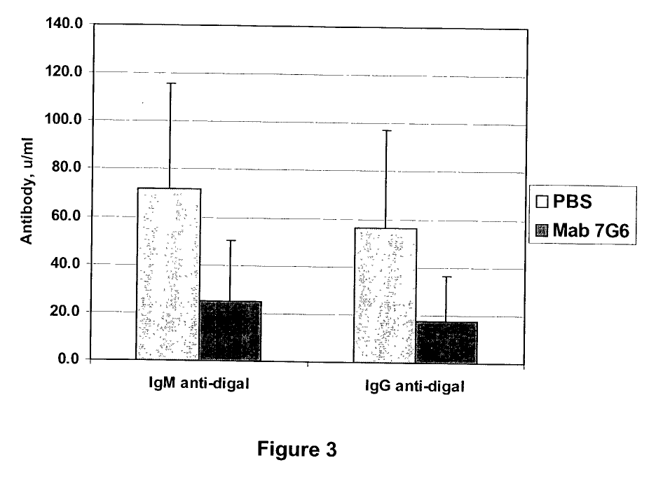

Treatment with 7G6 blocked the increase in anti-digal antibodies stimulated by RRBC immunization. The results of these experiments are summarized in Table 3 and FIG. 3.

Table 3: Levels of IgM and IgG anti-digal. in control and anti-CD21 treated mice

TABLE 3

| Animal # | Treatment | IgM anti-digal, u/ml | IgG anti-digal, u/ml |
|---|---|---|---|
| B1 | PBS | 82.9 | 65.0 |
| B2 | PBS | 15.4 | 9.9 |
| B3 | PBS | 9.7 | 7.1 |
| D1 | PBS | 117.6 | 47.2 |
| D2 | PBS | 12.8 | 8.8 |
| E1 | PBS | 97.5 | 86.9 |
| E2 | PBS | 97.5 | 74.7 |
| E3 | PBS | 99.4 | 75.4 |
| E4 | PBS | 119.5 | 136.8 |
| E5 | PBS | 67.5 | 48.8 |
| Mean | PBS | 72.0 ± 43.6 | 56.1 ± 41.1 |
| C1 | Mab 7G6 | 20.2 | 13.4 |
| C2 | Mab 7G6 | 13.9 | 8.8 |
| C3 | Mab 7G6 | 5.6 | 4.5 |
| F1 | Mab 7G6 | 92.9 | 69.7 |
| F2 | Mab 7G6 | 30.2 | 20.3 |
| G1 | Mab 7G6 | 5.6 | 6.4 |
| G2 | Mab 7G6 | 22.4 | 14.8 |
| G3 | Mab 7G6 | 18.2 | 12.6 |
| H1 | Mab 7G6 | 25.5 | 14.9 |
| H2 | Mab 7G6 | 15.6 | 8.8 |
| Mean | Mab 7G6 | 25.0 ± 25.1 | 17.4 ± 19.0 |

There was a 65% reduction in IgM anti-digal antibody levels in the antibody treated group compared to the PBS treated group. There was a 69% reduction in IgG anti-digal antibody levels in the antibody treated group compared to the PBS treated group.

### Example 5

### Use of Anti-CD21 Antibody in a Spontaneous Model of Autoimmune Thyroiditis

The NOD.H-2h4 strain of mice develops spontaneous autoimmune thyroiditis after receiving sodium iodide in drinking water. The development of autoimmune thyroiditis is accompanied by an increase in the level of antibodies to murine thyroglobulin (Braley-Mullen, H. and Yu, S. (2000) J. Immunol. 165: 7265-7269).

Animals are induced to develop thyroiditis by the introduction of sodium iodide in drinking water and then treated with 7G6 rat IgG2b anti-mouse CD21 antibody or a rat IgG2b isotype control (Kinoshita et al. 1990 Int. Immunol. 2: 651-659.

Treatment Protocols:

Each study consists of groups of animals including:

(a) control group that receives no treatment (saline).

(b) control group that receives isotype control antibody.

(c) test group that receives 7G6 anti-CD21.

At the age of 8 weeks, mice receive 0.05% sodium iodide in their drinking water to induce thyroiditis. In initial experiments, treatment of mice begins at 8 weeks of age (i.e., before onset of disease). Treatment regimes are detailed below. Blood samples are collected at 16 weeks of age and assayed for antibodies to thyroglobulin. Eight weeks after the introduction of sodium iodide into the drinking water, mice are sacrificed and thyroid tissue collected for histological evaluation. Thyroid lesions reach their maximum severity in NOD.H-2h4 mice 7-9 weeks after the introduction of sodium iodide in the drinking water.

In later experiments, treatment of the mice begins after 16 weeks of age, i.e., following the onset of disease. Mice are treated for four weeks using the regimes described below. Blood samples are then be collected to determine levels of anti-thyroglobulin antibodies. Mice are sacrificed and thyroid tissues are collected for histological evaluation.

Within each group, mice are treated with one of the following regimens:

1. Anti-CD21 antibody 500 μg via the tail vein twice weekly (day 3 and day 7).

2. Rat IgG2b antibody 500 μg via the tail vein twice weekly (day 3 and day 7).

3. Saline via the tail vein twice weekly (day 3 and day 7).

A minimum of 10 mice per group are assessed. The follow assays are used to follow the development of thyroiditis:

1. Anti-thyroglobulin ELISA. Titers of anti-thyroglobulin antibodies in animal sera are determined using a solid-phase ELISA.

2. Thyroid pathology is determined by histology. Damage to the thyroid follicles will be quantitated by measuring the degree of infiltration by mononuclear cells.

### Example 6

### Generation of Monoclonal Antibody 2B 12

Balb/cJ females were primed intramuscularly (i.m.) with 10 μg soluble human CD21 (domains 1-2)-Ig fusion protein (Hebell, T et al. (1991) Science 254:102-105) in Immuneasy adjuvant (Qiagen, Valencia, Calif.). Two weeks later, the mice were boosted with the same antigen and adjuvant combination. Six weeks later, the mice were boosted with 50 μg of sCD21 (1-2)-Ig in sterile PBS intravenously (i.v.). Three days later, the mice were sacrificed and spleen cells were fused with mouse myeloma cell line SP2/0 by standard procedures. After two weeks of selection, hybridoma supernatants were initially screened by direct ELISA with sCD21 (1-2)-Ig and then by the methods described in Examples 7-11.

### Example 7

### Disruption of CD21/C3d Interaction by mAb 2B12

Coating of BSA- or C3d-Microspheres

A volume equal to ˜1.5×10¹⁰ of a suspension of 1 μm carboxylate-modified fluorescent microspheres (# F8823, Molecular Probes, Eugene, Oreg.) was pelleted at 5000× g for 5 minutes, washed once in 1 ml 50 mM MES, pH 7.0 (2-[N-morpholino] ethanesulfonic acid) and resuspended in 0.5 ml 50 mM MES. To this suspension, 2 mg each of EDC (1-ethyl-3-(3-dimethylaminopropyl) carbodiimide HCl, Pierce # 22980) and sulfo-NHS (N-hydroxysulfosuccinimide Pierce # 24510) were added and the sample was then rotated in the dark for 20 minutes at room temperature. The activated microsphere suspension was spun at 5000× g for 5 minutes and washed three times in 1 ml 50 mM MES, pH 7.0. One volume of activated microsphere suspension was then added to 2 volumes of either C3d or BSA at 1 mg/ml protein in PBS, and rotated in the dark for 2 hours at room temperature. Microspheres were spun again and washed three times in 50 mM MES, pH 7.0. C3d- or BSA-coated microspheres were finally resuspended at 4×10⁹/ml in 50 mM HEPES, pH 7.4 containing 1% BSA and 0.01% NaN₃, and stored at 4° C.

Raji Cell-C3d Microsphere Binding Assay

Cells of the CD21⁺ human Burkitt's lymphoma cell line Raji (ATCC # CCL 86) were harvested from culture and washed in PFN (PBS containing 1% (v/v) fetal bovine serum (FBS: Gibco BRL, Gaithersburg, Md.) and sodium azide at 0.01% (w/v) to prevent CD21 surface modulation). Cells were resuspended in PFN at 10⁶/ml and 200 μl/sample aliquoted into FACS tubes. Mouse anti-human CD21 mAb 2B 12 was added in 50 μL PFN (30-50 ng as determined in preliminary experiments). Control tubes contained either no antibody or 30-50 ng of the mouse anti-human CD21 mAb HB5 (BD Pharmingen, La Jolla, Calif.), that recognizes an epitope on domains 3-4 of CD21 and has a minor effect on C3d binding. Tubes were incubated for 15 minutes at 4° C. BSA- or C3d-coated 1 μm fluorescent microspheres were then added to cells to give a final ratio of 25, 50 or 100 microspheres per Raji cell. The total volume of added microspheres was 20 μL. Tubes were vortexed briefly and incubated at room temperature for 20 minutes. PFN was added to give a final volume of 500 μL, and samples were then vortexed and analyzed at room temperature on a FACSCalibur flow cytometer. Raji-microsphere conjugates were identified in FL3 as a series of peaks representing cells bound to differing numbers of microspheres. For quantitative analysis of results a marker was set on histograms such that the first peak was excluded from the analysis.

The table below shows typical data of the percentage of Raji cells bound by C3d- or BSA-coated beads in the presence or absence of mAb 2B12, at varying microsphere:cell ratios.

It can be seen that addition of the mAb 2B12 completely inhibits C3d-specific binding to Raji cells. A small degree of inhibition by mAb HB5, especially at lower microsphere:cell ratios, was observed.

| | | % Cells | % Inhibition |
|---|---|---|---|
| | Condition | Bound* | by mAb** |
| 1 | Raji alone | 0 | |
| 2 | + BSA beads @ 25:1 | 1.5 | |
| 3 | + BSA beads @ 50:1 | 6.83 | |
| 4 | + BSA beads @ 100:1 | 22.55 | |
| 5 | + C3d beads @ 25:1 | 21.96 | |
| 6 | + C3d beads @ 50:1 | 50.04 | |
| 7 | + C3d beads @ 100:1 | 74.2 | |
| 8 | + C3d beads @ 25:1 + 30 ng 2B12 | 2.27 | 89.7 |
| 9 | + C3d beads @ 50:1 + 30 ng 2B12 | 5.77 | 88.5 |
| 10 | + C3d beads @ 100:1 + 30 ng 2B12 | 10.94 | 85.3 |
| 11 | + C3d beads @ 25:1 + 15 ng 2B12 | 5.96 | 72.9 |
| 12 | + C3d beads @ 50:1 + 15 ng 2B12 | 16.87 | 66.3 |
| 13 | + C3d beads @ 100:1 + 15 ng 2B12 | 37.92 | 48.9 |
| 14 | + C3d beads @ 25:1 + 30 ng HB5 | 15.44 | 29.7 |
| 15 | + C3d beads @ 50:1 + 30 ng HB5 | 39.8 | 20.5 |
| 16 | + C3d beads @ 100:1 + 30 ng HB5 | 67.25 | 9.4 |
| 17 | + C3d beads @ 25:1 + 15 ng HB5 | 19.36 | 11.8 |
| 18 | + C3d beads @ 50:1 + 15 ng HB5 | 42.38 | 15.3 |
| 19 | + C3d beads @ 100:1 + 15 ng HB5 | 69.35 | 6.5 |

*% Cells bound are % cells within marker M1
**% Inhibition = 100 × [(% Cells in M1 without Ab) − (% Cells in M1 with Ab)/% Cells in M1 without Ab]

### Example 8

### Detection of 2B12 Binding to Soluble Recombinant Human CD21 Protein Domains by Direct ELISA.

Microtiter wells were coated overnight at 4° C. with recombinant soluble CD21 (1-2)-Ig fusion protein (Hebell, ibid), or with recombinant soluble CD21 (1-4)-his, at 5 μg/ml. The recombinant soluble CD21 (1-4)-his was made as follows:

A fragment containing cDNA encoding the signal peptide and first four amino-terminal protein domains of human CD21 was amplified by PCR from human spleen total RNA (Clontech, Palo Alto, Calif.) using standard methods. The amplification primers were 5′CGCGAGCTCTTAGTGGTGGTGGTGGTGGTGAATTTCTTCACA-3′ (reverse) (SEQ ID NO:1) and 5′-GATCTTATAAATATGGGCGCCGCGGGCCTG-3′ (forward) (SEQ ID NO:2). The reverse primer encodes a (His)₆ purification tag followed by a stop codon and a Sac I restriction site. The forward primer encodes a Bg1 II restriction site. The amplified fragment was cloned into the pCR2. 1-TOPO® plasmid vector according to the manufacturer's instructions (Invitrogen, Carlsbad, Calif.). The nucleic acid sequence of the cloned fragment (SEQ ID NO:3) was confirmed using the Retrogen primer extension sequencing service (San Diego, Calif.). CD21 coding sequence is indicated in lower case letters; amplification primer sequences are in capital letters; Bgl II and Sac I restriction sites are underlined.

| | |
|---|---|
| 5′-AGATCTTATAAATatgggcg ccgcgggcct | (SEQ ID NO:3) |
| gctcgqggtt ttcttggctc tcgtcgcacc | |
| gggggtcctc gggatttctt gtggctctcc | |
| tccgcctatc ctaaatggcc ggattagtta | |
| ttattctacc cccattgctg ttggtaccgt | |
| gataaggtac agttgttcag gtaccttccg | |
| cctcattgga gaaaaaagtc tattatgcat | |
| aactaaagac aaagtggatg gaacctggga | |
| taaacctgct cctaaatgtg aatatttcaa | |
| taaatattct tcttgccctg agcccatagt | |
| accaggagga tacaaaatta gaggctctac | |
| acoctacaga catggtgatt ctgtgacatt | |
| tgcctgtaaa accaacttct ccatgaacgg | |
| aaacaagtct gtttggtgtc aagcaaataa | |
| tatgtggggg ccgacacgac taccaacctg | |
| tgtaagtgtt ttccctctcg agtgtccagc | |
| acttcctatg atccacaatg gacatcacac | |
| aagtgagaat gttggctcca ttgctccagg | |
| attgtctgtg acttacagct gtgaatctgg | |
| ttacttgctt gttggagaaa agatcattaa | |
| ctgtttgtct tcgggaaaat ggagtgctgt | |
| cccccccaca tgtgaagagg cacgctgtaa | |
| atctctagga cgatttccca atgggaaggt | |
| aaaggagcct ccaattctcc gggttggtgt | |
| aactgcaaac tttttctgtg atgaagggta | |
| tcgactgcaa ggcccacctt ctagtcggtg | |
| tgtaattgct ggacagggag ttgcttggac | |
| caaaatgcca gtatgtgaag aaattCACCA | |
| CCACCACCAC CACTAAGAGC TC-3′ | |

The cloned nucleic acid sequence (SEQ ID NO:4) contains an open reading frame encoding the CD21 sequence below. The putative signal peptide and the (His)₆ purification tag are underlined.

| | |
|---|---|
| MGAAGLLGVFLALVAPGVLGISCGSPPPIL NGRISY | (SEQ ID NO:4) |
| YSTPIAVGTVIRYSCSGTFRLIGEKSLLCITKDKVDG | |
| TWDKPAPKCEYFNKYSSCPEPIVPGGYKIRGSTPYRH | |
| GDSVTFACKTNFSMNGNXSVWCQANNMWGPTRLPTCV | |
| SVFPLECPALPMIHNGHHTSENVGSJAPGLSVTYSCE | |
| SYLVGEKIINCLSSGKWSAVPPTCEEARCKSLGRFPN | |
| GKVKEPPILRVGVTANFFCDEGYRLQGPPSSRCVIAG | |
| QGVAWTKMPVCEEIHHHHHH | |

The restriction fragment in SEQ ID NO:3 was released from pCR2.1-TOPO using Bg1 II and Sac I restriction endonucleases and was subcloned using standard techniques to the expression vector pBacPAK8 (Clontech, Palo Alto, Calif.), which had been linearized with the same restriction endonucleases. The nucleic acid sequence of the CD21 fragment in the resulting expression plasniid, CD21 SCRI-4, was confirmed using the Retrogen primer extension sequencing service (San Diego, Calif.).

CD21SCR1-4 was transfected into Sf9 insect cells using the BaculoPlatinum™ cotransfection vector (Orbigen, San Diego, Calif.) according to the manufacturer's instructions. Virus particles were harvested and a high titer stock was produced by reinfection of Sf9 cells according to standard methods. Recombinant protein SCD21(1-4)-his was produced by infection of TN5 insect cells with high titer virus, followed by incubation for approximately 50 hours. Recombinant sCD21(1-4)-his protein was harvested by nickel chelation chromatography on Ni-NTA agarose QIAGEN, Valencia, Calif.) according to the manufacturer's instructions. Further details are provided in Prodinger WM, et al., Immunopharmacology (1997) 39:141-8.

Relevant controls are the wells coated with mouse Ig Gλ (the isotype of the Ig fusion partner), or with an alternated his-tagged recombinant protein. After blocking wells with 1% BSA/PBS solution for 1 hour, wells were washed, test antibodies were added in duplicate and the plates incubated for 1 hour at room temperature. Plates were washed and a secondary goat anti-mouse Ig κ-alkaline phosphatase antibodies added to the wells for 30 minutes at room temperature. Plates were washed and developed in phenolphthalein monophosphate substrate (PPMP).

Typical OD results are:

| | sCD21-coated wells | MoIg-coated wells |
|---|---|---|
| Normal Mouse Serum 1:2500 | 0.055 | 0.061 |
| OKB7 (mo anti-huCD21) | 0.44 | 0.096 |
| 1:2500 | | |
| 2B12 culture supernatant | 0.65 | 0.045 |
| Control culture medium | 0.041 | 0.041 |

### Example 9

### Binding of 2B12 to Raji Cells Detected by Cell ELISA.

Raji cells were washed once in HFN (Hanks Balanced Salt Solution (HBSS) containing 1% v/v FBS and 0.05% w/v NaN₃) and aliquoted into microtiter wells. All steps were carried out at 4° C. The plates were spun, and supernatants removed. Test antibodies were added to wells, the plates were shaken and incubated for 30 minutes. Cells were then washed by 4 cycles of addition of HFN followed by spinning and removal of supernatants. Secondary alkaline-phosphatase-conjugated goat anti-mouse IgG (Jackson ImmunoResearch, West Grove, Pa.) was added in HFN, the plates were shaken and incubated for 30 minutes. Cells were washed again, as described above. PPMP substrate solution was added for 30 minutes, before plates were read at 550 nm to determine OD.

Typical OD results were:

| Addition | OD 550 nm |
|---|---|
| Control culture supernatant | 0.092 |
| 2B12 supernatant | 2.451 |
| Mouse IgG | 0.072 |
| Anti-hu CD21 mAb BE-5 | 0.546 |
| Anti-hu CD21 mAb Bly-4 | 1.171 |

### Example 10

### Binding of 2B12 to Raji Cells Detected by Flow Cytometry

Raji cells were washed in cold PFN (PBS containing 1% FBS and 0.1% NaN3) and aliquoted into FACS tubes at 2×10⁵/sample. Test samples were added to cells (e.g., hybridoma supernatants, purified mAb), mixed and incubated at 4° C. for 20 minutes. Cells were washed in PFN and a secondary fluorophore-conjugated Ab was added (e.g., goat anti-mouse IgG (H+L)-FITC Jackson ImmunoResearch, West Grove, Pa.). Cells were again incubated at 4° C. for 20 minutes and then washed. Dead cells were excluded from the flow cytometric analysis by addition of propidium iodide to a final concentration of 2 μg/ml in PFN. Cells were then analyzed on a FACSCalibur, and intensity of staining measured by MFI. Typical results are shown below:

| Condition | Mean Fluorescent Index (MFI) |
|---|---|
| Raji alone | 5 |
| + control culture medium | 4 |
| + 2B12 culture supernatant | 165 |
| + OKB7* | 123 |
| + BE-5** | 85 |
| + HB5*** | 117 |

*OKB7 from Ortho Diagnostics (Raritan, NJ)
**BE-5 from BioSource International (Camarillo, CA)
***HB5 from BD Pharmingen (La Jolla, CA)

### Example 11

### Immunoprecipitation of CD21 from Raji Cells by 2B12

Cells of the human B lymphoblastoid line Raji were surface biotinylated and solubilized in detergent-containing buffer. Cell lysates were incubated with culture supernatants from the myeloma cell line SP2/0 (negative control), or from the hybridoma HB5 (mouse anti-human CD21 domains 3-4), or from test hybridoma 2B12. Subsequently, the lysates were incubated with a rabbit anti-mouse polyclonal antibody, followed by Protein-A bearing killed Staph. aureus organisms (Pansorbin™), to collect immune complexes. The pellets from these primary immunoprecipitations were resolved by SDS-PAGE, transferred to a membrane, developed with peroxidase-conjugated streptavidin and then revealed by chemiluminescence.

To demonstrate that the known anti-CD21 mAb and the test 2B 12 antibody recognize the same molecule, the supernatants from the primary immunoprecipitation were each divided into three parts, and subjected to a second round of immunoprecipitation using the same antibodies.

The results of this experiment unequivocally demonstrated that the mAb 2B 12 recognizes the same molecule as mAb HB5. First, they both directly immunoprecipitated a molecule of similar size ˜140 kD. Secondly, the supernatant depleted of CD21 by mAb HB5 is also depleted of the molecule recognized by mAb 2B 12. Conversely, the supernatant depleted of the molecule recognized by 2B 12 is also depleted of CD21 immunoprecipitated by HB5. Thus, mAb HB5 and 2B 12 both immunoprecipitate CD21 from Raji cells.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

What is claimed is:

1. A method for treating an individual suffering from an antibody-mediated pathology comprising administering to said individual an effective amount of an agent which interferes with C3d binding to CD21, whereby a symptom of the antibody-mediated pathology is ameliorated.

2. The method according to claim 1 wherein the antibody-mediated pathology is an autoimmune disease.

3. The method according to claim 2 wherein the autoimmune disease is systemic lupus erythematosus.

4. The method according to claim 1 wherein the antibody-mediated pathology is xenotransplantation rejection.

5. The method according to claim 1 wherein the antibody-mediated pathology is thyroiditis.

6. The method according to claim 1 wherein the agent is an antibody which specifically binds to a region of CD21 to which C3d binds.

7. The method according to claim 6 wherein said region comprises short consensus region 1 and short consensus region 2.

8. The method according to claim 6 wherein said region comprises short consensus region 1 or a portion thereof.

9. The method according to claim 6 wherein said region comprises short consensus region 2 or a portion thereof.

10. The method according to claim 1 wherein said agent is an antibody.

11. A method for delaying development of a symptom associated with an antibody-mediated pathology in an individual comprising administering to said individual an effective amount of an agent which interferes with C3d binding to CD21, wherein development of a symptom of the antibody-mediated pathology is delayed.

12. The method according to claim 11 wherein the antibody-mediated pathology is an autoimmune disease.

13. The method according to claim 12 wherein the autoimmune disease is systemic lupus erythematosus.

14. The method according to claim 11 wherein the antibody-mediated pathology is xenotransplantation rejection.

15. The method according to claim 11 wherein the antibody-mediated pathology is thyroiditis.

16. The method according to claim 11 wherein the agent is an antibody which specifically binds to a region of CD21 to which C3d binds.

17. The method according to claim 16 wherein said region comprises short consensus region 1 and short consensus region 2.

18. The method according to claim 16 wherein said region comprises short consensus region 1 or a portion thereof.

19. The method according to claim 16 wherein said region comprises short consensus region 2 or a portion thereof.

20. The method according to claim 11 wherein said agent is an antibody.
